(19) United States
(12) Patent Application Publication
(10) Pub. No.: US 2026/0209771 A1
(43) Pub. Date: Jul. 23, 2026

(54) PROGRAMMABLE RNA EDITING IN PARKINSON'S DISEASE THERAPY

(71) Applicant: The Board of Trustees of the Leland Stanford Junior University, Stanford, CA (US)

(72) Inventors: Jin Billy Li, Stanford, CA (US); Rohini Datta, Stanford, CA (US)

(21) Appl. No.: 19/140,121

(22) PCT Filed: Dec. 19, 2023
(86) PCT No.: PCT/US2023/084873

§ 371 Date: Jun. 17, 2025

Related U.S. Application Data
(60) Provisional application No. 63/433,542, filed on Dec. 19, 2022.

(51) Int. Cl. C12N 15/113; A61P 25/28

(52) U.S. Cl. CPC C12N 15/1137; A61P 25/28; C12N 2310/11; C12N 2310/20; C12N 2310/315; C12N 2310/321; C12N 2310/322

(57) ABSTRACT

The present disclosure relates to an antisense oligonucleotide suitable for editing an adenosine in a target RNA nucleic acid sequence to inosine (A-to-I editing), wherein the antisense oligonucleotide comprises a sequence that is substantially complementary to the target RNA nucleic acid sequence: 5′-A C U A C A G C A U U G C-3′ (SEQ ID NO: 1), or complementary to a sequence that has at least 80% sequence identity to SEQ ID NO: 1, wherein A is the target adenosine to be edited in the target nucleic acid; and the antisense oligonucleotide can be represented by: 3′-X₋₅ X₋₄ X₋₃ X₋₂ X₋₁ X₀ X₊₁ X₊₂ X₊₃ X₊₄ X₊₅ X₊₆ X₊₇-5′, wherein X represents a nucleic acid, X₀ represents cytosine (C), deoxycytosine (dC), or uracil (U) directly opposite to the target adenosine to be edited, and wherein there is a mismatch between the antisense oligonucleotide and the target nucleic acid at one or more of the following position(s): a) −5 (X₋₅); b)+3 (X₊₃); c)+7 (X₊₇); d)+7 (X₊₇) and +2 (X₊₂); e)+7 (X₊₇) and −5 (X₋₅); or f)+6 (X₊₆) and −5 (X₋₅).

### PRIORITY STATEMENT

This application claims priority to U.S. Provisional Patent Application No. 63/433,542, filed Dec. 19, 2022, the entire contents of which are incorporated herein by reference in its entirety for all purposes.

### SEQUENCE LISTING

The text of the computer readable sequence listing filed herewith, titled “STDU2-41485-601_SQL”, created Dec. 19, 2023, having a file size of 102,273 bytes, is hereby incorporated by reference in its entirety.

### FIELD

The present invention relates to the field of medicine, in particular to the field of site-directed A-to-I RNA editing, whereby an LRRK2 encoding RNA sequence is targeted by an antisense oligonucleotide (ASO). Provided herein are ASOs targeting Irrk2 transcripts, compositions and vectors comprising said ASOs, and methods of their use. The present disclosure also relates to high-throughput screening methods and guide RNAs for use in site-directed RNA editing. In particular, provided herein are high-throughput screening methods for identifying guide RNAs (gRNAs) effective for site directed RNA editing of the pathogenic G2019S mutation in the LRRK2 kinase known to cause Parkinson's Disease (PD), and to guide RNAs capable of efficient site-directed RNA editing of this mutation.

### BACKGROUND

Parkinson's Disease (PD) is one of the most common neurodegenerative disorders in the world initially described in the 1800's by James Parkinson as the ‘Shaking Palsy’ (Mhyre et al., (2012)). PD is generally classified by somatic symptoms including resting tremors, rigidity, bradykinesis, and postural problems. However, to date there are no approved disease-modifying therapies. Studies in PD families have led to the identification of 15 PD loci, and 11 genes. Although follow-up genetic studies have been inconsistent for some of them or conclusive data are still pending, there is evidence that five of those genes (α-synuclein, parkin, PTEN-induced putative kinase 1, DJ-1, and leucine-rich repeat kinase 2 (lrrk2)) cause typical PD (Coppedè, 2012). Specifically, mutations in the gene encoding LRRK2 have been shown to be associated with autosomal dominant PD and are the most common genetic cause of both familial and sporadic PD (Li et al. (2014)). In fact, LRRK2 accounts for 4% of familial PD and 1% of sporadic PD across all populations (Kumari et al., 2009).

The product of the leucine-rich repeat kinase 2 (Irrk2) gene is a highly conserved large 286-kDa protein. The LRRK2 protein contains two enzymatic domains (a GTPase and a kinase domain), multiple protein-protein interaction domains, including a leucine-rich repeat (LRR), a WD40 repeat, and a LRRK2 specific repeat domain. The LRRK2 interaction domains are thought to serve as protein binding modules where LRRK2 acts as a signaling scaffold, while LRRK2 GTPase and kinase enzyme activities are important in regulating LRRK2 dependent cellular signaling pathways.

Up to now, established dominant inherited PD-associated mutations in LRRK2 include G2019S, R1441C/G/H, Y1699C, I2020T and N1437H (Bardien S, et al., (2011)). Specifically, the G2019S mutation is a G to A point mutation at the nucleotide level and the most common cause of hereditary PD. The autosomal dominant G2019S mutation occurs in the kinase domain of LRRK2 and increases the kinase activity by twofold (Alessi and Sammler, 2018). Rab GTPases, which drive membrane trafficking, have been implicated as key targets of LRRK2 kinase (Steger et al., 2016, 2017).

No cure is currently available for the neurodegeneration observed in PD. While several promising small-molecule inhibitors of LRRK2 have recently entered clinical trials (Jennings et al., 2022), some of these inhibitors continue to pose risks of toxicity due to inhibition of normal LRRK2 function in membrane trafficking, autophagy and inflammation and associated side effects (Baptista et al., 2013; Fuji et al., 2015). Accordingly, what is needed are alternative treatment options that restore normal LRRK2 function without the risk of excessive inhibition.

ite-Directed RNA Editing (SDRE) describes the alteration of an RNA sequence by introducing or removing nucleotides from an RNA or by changing the character of a nucleobase by deamination. RNA editing enzymes are known in the art. The first RNA editing process discovered in mammals was the deamination of cytidine (C) by APOBEC proteins to form uridine (U)(Zinshteyn and Nishikura, 2009). To date, the two most useful and most studied types of RNA editing are cytidine (C) to uridine (U)(“C-to-U”) and adenosine (A) to inosine (I)(“A-to-I”) conversions. Notably, for therapeutic purposes and the most prevalent type of RNA editing in higher cukaryotes is the “A-to-I” conversion.

“A-to-I” conversion is catalysed by the adenosine deaminases acting on RNA (ADARs) family. “A-to-I” editing was first identified in Xenopus eggs (Bass and Weintraub, 1987; Rebagliati and Melton, 1987). Human cDNA encoding “double stranded RNA adenosine deaminase” was first cloned by Kim et al. (1994) and the adenosine to inosine (“A-to-I”) conversion activity of the protein confirmed by recombinant expression in insect cells. “A-to-I” editing changes the informational content of the RNA molecule, as inosine preferentially base pairs with cytidine and is therefore interpreted as guanosine (G) by the translational and splicing machinery. During this enzymatically catalyzed reaction adenosine is changed via a hydrated intermediate to inosine. While guanosine can form three hydrogen bonds to the complementary base cytidine, inosine can form only two hydrogen bonds to cytidine. The translational machinery reads inosine as a guanosine. Therefore, ADARs have the effect of introducing a functional adenosine to guanosine mutation on the RNA level. The ability of ADARs to alter the sequence of RNAs has also been used to artificially target RNAs in vitro in cells for RNA editing. Potentially this approach may be used to repair genetic defects and alter genetic information at the RNA level.

Over the years, three vertebrate ADAR genes have been identified, which give rise to several ADAR proteins that result from alternative promoters or represent splice variants (Wulff and Nishikura, 2010). These proteins are expressed across various types of human tissues, and which can alter splicing and translation machineries, double-stranded RNA (dsRNA) structures and the binding affinity between RNA and RNA-binding proteins (Tomaselli et al., 2014; Zinshteyn and Nishikura, 2009). Of the three known ADAR genes, hADAR1 and hADAR2 are expressed in most tissues and encode active deaminases. Human ADAR3 (hADAR3) has been described to only be expressed in the central nervous system and reportedly has no deaminase activity in vitro. While all ADARs are multidomain proteins, comprising a targeting or dsRNA-binding domain (dsRBD) and a catalytic domain, ADAR1 proteins additionally comprise one or more Z binding domains, while splice variant ADAR2R and ADAR3 comprises an R domain (Zinshteyn and Nishikura, 2009; Wulff and Nishikura, 2010). Accordingly, in some embodiments, the ADAR is hADAR1, hADAR2 or hADAR3. “A-to-I” RNA editing systems employing endogenous adenosine deaminase enzymes have been extensively studied, i.e., the use of exogenous oligonucleotides to specifically recruit endogenous adenosine deaminases to a specific target site of a target RNA thereby providing an improved system for targeted RNA editing.

For instance, oligonucleotide constructs for site-directed RNA editing are described in patent applications WO 2016/097212 and WO 2017/010556, which utilize endogenous cellular pathways, i.e., endogenous ADAR, to edit endogenous RNA.

Antisense oligonucleotides (ASOs) are generally short single-stranded synthetic RNA or DNA molecules, which use Watson-Crick base pairing to bind sequence specifically to the target RNA. ASOs can work through many mechanisms depending, in part, on the region in the RNA sequence that is targeted and ASO design/chemical properties.

To ensure ASO specificity, their sequences are ideally complementary or at least partially complementary to the target RNA. However, in the case of site-directed mutagenesis, i.e., “A-to-I” RNA editing, the ASO targeting domain contains a mismatch opposite the targeted adenosine. It is to be noted that several endogenous substrates of ADAR contain mismatches and/or bulges (Thomas and Beal, 2017) and therefore could alter or even improve substrate recognition, if these features are mimicked in the ASO/resulting dsRNA. For instance, ADARs may have a preference for adenosines within a certain local sequence context. While most efficient editing seems to occur at adenosines found opposite C (C-A), A-U pairs are also commonly edited (Wong et al., 2001). WO 2017/220751 describes that additional mismatches in the formed dsRNA, caused by nucleotides in the oligonucleotide that do not form perfect base pairs with the target RNA are tolerable but not essential for specific targeted editing of the target RNA sequence.

ASOs can also be chemically modified to improve their properties. For instance, ASOs can be modified to protect them against nucleases and to increase their effectiveness. While phosphorothioate (PS) modifications seem to have a positive effect on ASOs stability and pharmacokinetics, the difference in chirality of PS linkages may have a substantial influence on the ASO's overall property. The use of ADAR-mediated RNA editing in non-human primates using stereopure chemically modified oligonucleotides has previously been reported (Monian et al., 2022). These oligonucleotides typically are very rich in 2′-F-modifications within the 5′ half, which are generally present as blocks of 2′-F-modifications and uniform block of 2′-O-Methyl-modifications within the 3′ terminus on either side of the CBT. Furthermore, these oligonucleotides contain massively stereopure PS-modified backbone and massively stereopure PS linkages and additional charge-neutral PN linkage (also stereopure), the latter of which is not yet applied in the clinics. Analogously, WO 2021/071858 relates to oligonucleotides comprising a first and second domain, wherein the first domain comprises one or more 2′-F modifications, and wherein the second domain comprises one or more sugars that do not have a 2′-F modification. WO 2022/099159 relates to oligonucleotides with a first and second domain, wherein the domains comprise specific percentages of 2′-F modifications and aliphatic substitutions.

The use of endogenous ADAR-mediated RNA editing in non-human primates using stereopure chemically modified oligonucleotides has previously been reported (Monian et al., 2022). These oligonucleotides typically are very rich in 2′-F-modifications within the 5′ half, which are generally present as blocks of 2′-F-modifications and uniform block of 2′-O-Methyl-modifications within the 3′ terminus on either side of the CBT. Furthermore, these oligonucleotides contain massively stereopure PS-modified backbone and massively stereopure PS linkages and additional charge-neutral PN linkage (also stereopure), the latter of which is not yet applied in the clinics. That precise, site-specific RNA editing can be achieved by recruiting endogenous ADARs with antisense oligonucleotides has previously been shown by Merkle et al. (2019). Merkle et al. (2019) were able to demonstrate that chemically optimized ASOs can be used to recruit endogenous human ADARs to edit endogenous transcripts in a simple and programmable way with almost no off-target editing.

In WO 2020/001793, artificial nucleic acids for “A-to-I” editing were provided, wherein the nucleic acid comprises a targeting sequence and recruiting moiety. Similarly, WO 2018/041973 relates to ASOs that can bring about specific editing of adenosines in a target RNA sequence, wherein said ASO does, however, not form an intramolecular hairpin or stem-loop structure. WO 2018/041973 relates to chemically modified single-stranded RNA-editing oligonucleotides for the deamination of a target adenosine by an ADAR enzyme whereby the central base triplet (CBT) of three sequential nucleotides comprises a sugar modification and/or a base modification. It was found that deoxyribose at all three positions of the CBT is well tolerated and provides substantial stabilization against nuclease digestion.

ASO-based therapies have been gaining more traction over the past years for the treatment of different medical conditions and diseases, and in specific genetic disorders, including Parkinson's Disease. For instance, WO 2021/231673A 1 relates to methods and compositions for editing an LRRK2 polynucleotide, e.g., an LRRK2 polynucleotide comprising a SNP associated with Parkinson's Disease (PD). Specifically, WO 2021/231673A1 relates to a method wherein a LRRK2 polynucleotide carrying a single nucleotide polymorphism (SNP) is contacted with a guide oligonucleotide to mediate an adenosine to inosine alteration of the SNP using adenosine deaminase acting on RNA (ADAR). WO 2012/242903A2 relates to compositions and methods that can be used to treat disease or conditions that arise from genomic mutations and uses engineered polynucleotides to target RNA encoding the LRRK2 polynucleotide.

Despite being a promising technology, due to difficulties pertaining to, e.g., stability and cellular delivery, toxicity and off-target effects, few ASOs have been marketed to date. The success of ASOs is, in part, defined by their ability to affect the target. Hence, to translate ASO-based therapies into a widespread clinical success, one aim is to improve selectivity towards the target, reduce the off-target effects and improve ADAR recruitment. Specifically, there exists an ongoing need for novel and improved antisense oligonucleotides and methods that can selectively and efficiently edit target nucleic acids encoding LRRK2 to modify the sequence or correct any pathogenic mutations associated with the Irrk2 gene in order to treat and/or prevent Parkinson's Disease (PD).

### SUMMARY

Provided herein are antisense oligonucleotides suitable for editing an adenosine in a target RNA nucleic acid sequence to inosine (A-to-I editing), wherein the antisense oligonucleotide comprises a sequence that is substantially complementary to the target RNA nucleic acid sequence: 5′-A C U A C A G C A U U G C-3′ (SEQ ID NO: 1), or complementary to a sequence that has at least 80% sequence identity to SEQ ID NO: 1, wherein A is the target adenosine to be edited in the target nucleic acid; and the antisense oligonucleotide can be represented by: 3′-X₋₅ X₋₄ X₋₃ X₋₂ X₋₁ X₀ X₊₁ X₊₂ X₊₃ X₊₄ X₊₅ X₊₆ X₊₇-5′, wherein X represents a nucleic acid, X₀ represents cytosine (C), deoxycytosine (dC), or uracil (U) directly opposite to the target adenosine to be edited, and wherein there is a mismatch between the antisense oligonucleotide and the target nucleic acid at one or more of the following position(s): a) −5 (X₋₅); b)+3 (X₊₃); c)+7 (X₊₇); d)+7 (X₊₇) and +2 (X₊₂); e)+7 (X₊₇) and −5 (X₋₅); or f)+6 (X₊₆) and −5 (X₋₅).

Also provided herein is an antisense oligonucleotide suitable for editing an adenosine in a target RNA nucleic acid sequence to inosine (A-to-I editing), wherein the antisense oligonucleotide comprises a sequence that is substantially complementary to the target RNA nucleic acid sequence: 5′-U G C U G A C U A C A G C A U U G C-3′ (SEQ ID NO: 2), or complementary to a sequence that has at least 80% sequence identity to SEQ ID NO: 2, wherein A is the target adenosine to be edited in the target nucleic acid; and the antisense oligonucleotide can be represented by: 3′-X₋₁₀ X₋₉ X₋₈ X₋₇ X₋₆ X₋₅ X₋₄ X₋₃ X₋₂ X₋₁ X₀ X₊₁ X₊₂ X₊₃ X₊₄ X₊₅ X₊₆ X₊₇-5′, wherein X represents a nucleic acid, X₀ represents cytosine (C), deoxycytosine (dC), or uracil (U) directly opposite to the target adenosine to be edited, and wherein there is a mismatch between the antisense oligonucleotide and the target nucleic acid at one or more of the following position(s): g)+6 (X₊₆) and −5 (X₋₅); h)+7 (X₊₇) and −10 (X₋₁₀); or i)+3 (X₊₃), −5 (X₋₅), and −8 (X₋₈).

Also provided herein is a vector that comprises the antisense oligonucleotide of the invention.

Also provided herein are pharmaceutical compositions comprising an antisense oligonucleotide of the invention or a vector of the invention. In some aspects, provided herein methods for site-directed A-to-I editing of a target RNA, comprising providing to a cell or subject an oligonucleotide of the invention, a vector of the invention or a pharmaceutical composition of the invention. Further, the invention provides for an in vitro method for deaminating at least one specific adenosine present in a target RNA sequence in a cell. Also provided herein are methods of treating a subject suffering from a genetic disorder, condition, or disease associated with the Irrk2 gene, wherein the method comprises administering an oligonucleotide, a vector or pharmaceutical composition of the invention. In other aspects, provided herein are fusion constructs comprising a guide RNA sequence and a target sequence. In some embodiments, the guide RNA sequence comprises a recruitment domain and an antisense sequence that is complementary (e.g., substantially complementary, or perfectly complementary) to the target sequence. In some embodiments, the target sequence is derived from the Irrk2 gene having a G2019S mutation. The fusion constructs described herein can be evaluated in a high throughput screen to identify constructs with highest RNA editing efficacy. Other aspects and embodiments of the disclosure will be apparent in light of the following detailed description and accompanying figures.

### BRIEF DESCRIPTION OF THE DRAWINGS

FIG. 1 is a schematic of an exemplary ADAR-recruiting gRNA.

FIG. 2A-2B show a prototype guide RNA design for editing LRRK2 G2019S. As shown, the guide RNA comprises an ADAR-recruitment domain and an antisense domain (FIG. 2A). For high-throughput screening of antisense sequences, the guide RNA comprising the recruitment domain and antisense domain are covalently linked to the target sequence, as shown in FIG. 2B, to generate a hairpin structure or prototype. This allows the identification of the editing at the target site as well as the corresponding guide RNA. The G-to-A mutation in the target sequence (e.g., LRRK2 target sequence) is shown in red. The A/C mismatch at the editing site is included as a known editing enhancer.

FIG. 3 shows a LRRK2 library design wherein the prototype was modified by randomizing the antisense sequence, as described herein, to generate a guide RNA library. The guide RNA library was used in a high-throughput screening method to identify optimized guide RNAs for efficient site-directed RNA editing of the LRRK2 G2019S mutation causing PD. Further, FIG. 3. describes a workflow that was used to identify and generate guide RNAs with the ability to correct the G-to-A point mutation (at the nucleotide level) in LRRK2 G2019S. The workflow involves screening guide RNAs in cultured cells expressing endogenous ADAR to identify top guide RNA candidates that show high editing by quantifying G: A ratios from Illumina sequencing

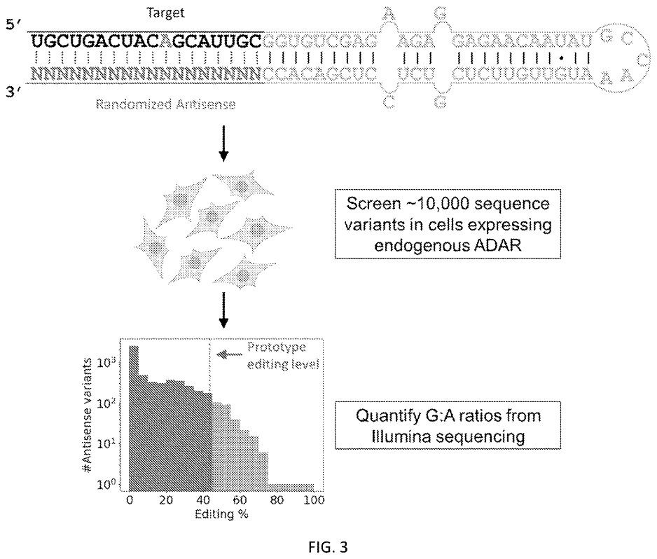

FIG. 4A is a heatmap showing positional enrichment between the antisense sequence and the target sequence that were shown to affect RNA editing positively. This is a cumulative effect of single, double, triple and multiple mismatches. FIG. 4B is a heatmap showing the effect of single mismatches between the antisense sequence and the target sequence on RNA editing.

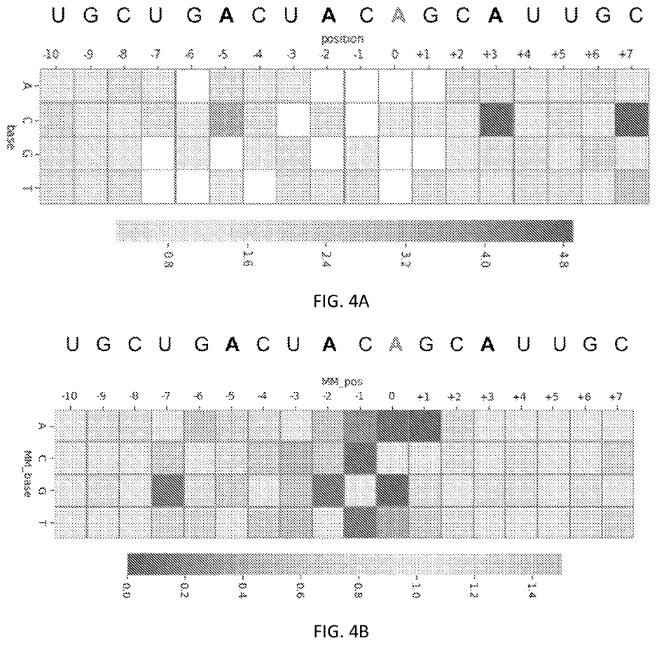

FIG. 5 shows the top six guide RNA sequences for editing LRRK2 G2019S with optimized antisense domains with highest on-target editing and lowest off-target editing levels.

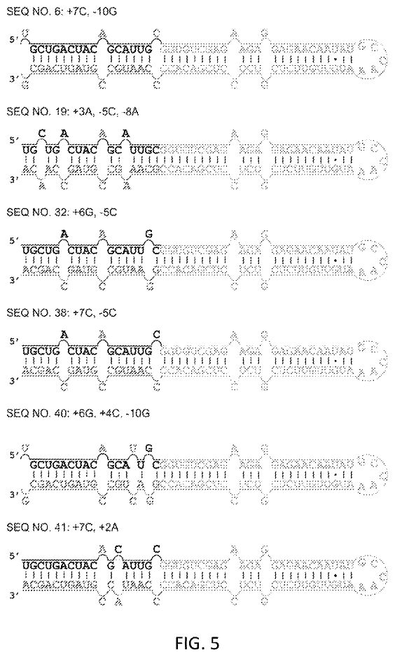

FIG. 6A shows an exemplary schematic to generate CLUSTER gRNAs to edit LRRK2 G2019S by addition of three recruitment sequences (RS #1, RS #2, RS #3) that are connected to each other by a three-nucleotide linker, AAA. Recruitment sequences enhance binding to target mRNA. The antisense sequence is optimized by addition of editing-enhancing mismatches identified in the high-throughput method described above. FIG. 6B shows editing levels of the CLUSTER gRNAs with optimized sequences that efficiently edit LRRK2 G2019S in FlpIn TRex 293 cells expressing the G2019S mutant. Here, the gRNAs are delivered into cells by transduction using AAV9 vectors that express a tdTomato reporter gene. This allows selection of cells that uptake the gRNA and editing thereof, using fluorescence activated cell sorting (FACS). FIG. 6C shows ability of the top candidate CLUSTER gRNA with the +7C, −10G mismatch to reduce hyperactivation of mutant LRRK2 kinase to potentially physiological levels.

FIG. 7 shows editing levels of optimized CLUSTER gRNAs in a clinically relevant cell type-neuronal progenitor cells differentiated from iPSCs derived from a PD patient carrying the LRRK2 G2019S mutation. These patients carry the mutation on both alleles. Here, the gRNAs are delivered into cells by transduction using adeno associated virus 9 (AAV9) vectors that express a tdTomato reporter gene. This allows selection of cells that uptake the gRNA and editing thereof.

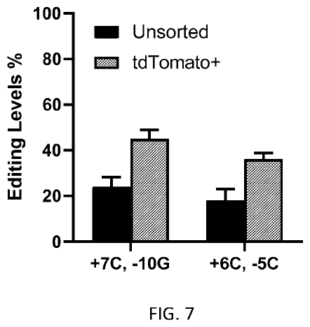

FIG. 8 shows an exemplary workflow to screen antisense variants and CLUSTER gRNAs in cultured cells. These two approaches can be combined to increase editing efficacy, which are tested in patient-derived cells.

### DETAILED DESCRIPTION

Provided herein are oligonucleotides that can harness the endogenous ADAR deaminase for efficient A-to-G RNA editing of the LRRK2 G2019S target sequence with high efficiency and specificity in cultured cells. Attachment of clusters of recruitment sequences was able to significantly boost editing levels of the identified guide RNAs. The guide RNAs disclosed herein can reduce pathogenic effects of mutant LRRK2 in cells, including reducing LRRK2 hyperactivation to potentially physiological levels. These guide RNAs are capable of efficiently editing the G2019S mutation in LRRK2 transcripts in pre-clinical models, such as human iPSC-derived neuronal progenitor cells derived from Parkinson's Disease patients that carry the mutation on both alleles. Also, provided herein are a high-throughput screening methods for identifying guide RNAs that are capable of efficient site-directed RNA editing of a pathogenic G2019S mutation in the LRRK2 kinase.

### 1. Definitions

To facilitate an understanding of the present technology, a number of terms and phrases are defined below. Additional definitions are set forth throughout the detailed description.

The articles “a” and “an” are used herein to refer to one or to more than one (i.e., to at least one) of the grammatical object of the article. By way of example, “an element” means one element or more than one element, e.g., a plurality of elements.

The terms “about” and “approximately” may be understood to permit standard variation as would be understood by those of ordinary skill in the art.

The terms “comprise(s)”, “include(s)”, “having”, “has”, “can”, “contain(s)”, and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising”, “consisting of” and “consisting essentially of”, the embodiments or elements presented herein, whether explicitly set forth or not.

For the recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. For example, any nomenclature used in connection with, and techniques of, cell and tissue culture, biochemistry, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “Pgly”), pipecolic acid, thioproline (“ThioP” or “tPro”), homoLysine (“hLys”), and homoArginine (“hArg”).

As used herein, the term “artificial” refers to compositions and systems that are designed or prepared, and are not naturally occurring. For example, an artificial peptide or nucleic acid is one comprising a non-natural sequence (e.g., a nucleic acid or a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine(S) and Threonine (T); and
8) Cysteine (C) and Methionine (M).

Naturally occurring residues may be divided into classes based on common side chain properties, for example: polar positive (or basic)(histidine (H), lysine (K), and arginine (R)); polar negative (or acidic)(aspartic acid (D), glutamic acid (E)); polar neutral (serine(S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

The terms “complementary” and “complementarity” refer to the ability of a nucleic acid to form hydrogen bond(s) with another nucleic acid sequence by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., 50%, 60%, 70%, 80%, 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., 65%, 70%, 75%, 80%, 85%, 90%, 95%, 97%, 98%, 99%, or 100%) over a region of at least 8 nucleotides (e.g., 9, 10, 11, 12, 13, 14, 15, 16, 17, 18, 19, 20, 21, 22, 23, 24, 25, 30, 35, 40, 45, 50, or more nucleotides), or if the two nucleic acid sequences hybridize under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., infra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Sambrook et al., Molecular Cloning: A Laboratory Manual, 3rd ed., Cold Spring Harbor Press, Cold Spring Harbor, N.Y. (2001); and Ausubel et al., Current Protocols in Molecular Biology, Greene Publishing Associates and John Wiley & Sons, New York (1994).

The term “adenosine deaminases acting on RNA” or “ADARs” are used herein to refer to a class of enzymes that naturally catalyze A-to-I editing of sites within double-stranded RNA (dsRNA) regions of the transcriptome of higher organisms. The I nucleotide is read as G by the ribosome during translation, that leads to A-to-G modifications in RNA. ADARs can play important roles in the regulation of protein function, RNA splicing, immunity and RNA interference.

The term “ADAR fusions” as used herein refers to engineered enzymes that comprise an ADAR deaminase domain and a domain which is able to bind a guide RNA.

The term “donor nucleic acid molecule” refers to a nucleotide sequence that is inserted into the target DNA (e.g., genomic DNA). As described above the donor DNA may include, for example, a gene or part of a gene, a sequence encoding a tag or localization sequence, or a regulating element. The donor nucleic acid molecule may be of any length. In some embodiments, the donor nucleic acid molecule is between 10 and 10,000 nucleotides in length. For example, between about 100 and 5,000 nucleotides in length, between about 200 and 2,000 nucleotides in length, between about 500 and 1,000 nucleotides in length, between about 500 and 5,000 nucleotides in length, between about 1,000 and 5,000 nucleotides in length, or between about 1,000 and 10,000 nucleotides in length.

The terms “hairpin”, “hairpin loop”, “stem loop”, and/or “loop” are used interchangeably herein to refer to a structure formed in a single stranded oligonucleotide when sequences within the single strand which are complementary when read in opposite directions base pair to form a region whose conformation resembles a hairpin or loop.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

As used herein, a “nucleic acid” or a “nucleic acid sequence” refers to a polymer or oligomer of pyrimidine and/or purine bases, preferably cytosine (C), thymine (T), and uracil (U), and adenine (A) and guanine (G), respectively. The present technology contemplates any deoxyribonuclcotide (DNA), ribonucleotide (RNA), or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506, incorporated herein by reference in their entireties), locked nucleic acid (LNA; see, Wahlestedt et al., Proc. Natl. Acad. Sci. U.S.A., 97:5633-5638 (2000), incorporated herein by reference in its entirety), cyclohexenyl nucleic acids (see, Wang, J. Am. Chem. Soc., 122:8595-8602 (2000), incorporated herein by reference in its entirety), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (i.e., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid”, “polynucleotide”, “nucleotide sequence”, and “oligonucleotide” are used interchangeably. They refer to a polymeric form of nucleotides of any length, either deoxyribonucleotides or ribonucleotides, or analogs thereof.

As used herein, the term “wobble base pair” is a pairing between two nucleotides that does not follow Watson-Crick base pair rules. The four main wobble base pairs are guanine-uracil (G-U), hypoxanthine-uracil (I-U), hypoxanthine-adenine (I-A), and hypoxanthine-cytosine (I-C).

As used herein, the term “chemical modification” preferably refers to a chemical modification selected from backbone modifications, sugar modifications or base modifications, including abasic sites. A “chemically modified oligonucleotide” in the context of the present invention may refer to a nucleic acid comprising at least one chemically modified nucleotide.

The term “modified” nucleobase” and “modified base” may be used interchangeably with the term “nucleobase”. Nucleobases may be modified or unmodified. Hence, in some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases. In one embodiment, the modified nucleobase is capable of increasing hydrogen bonding, base pair stacking interactions and/or stabilizing a nucleic acid complex. In another embodiment, the modified nucleobase (e.g., Benner's base) is capable of mimicking the N3 protonated cytosine base. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U. Modifications include but are not limited to nonstandard nucleobases 5-methyl-2′-deoxycytidine (m5C), pseudouridine (pU), dihydrouridine, inosine (I), and 7-methylguanosine. Other modifications may include nucleobase replacement by (N) heterocycles (e.g., nebularine) or aromatic rings that stack well in the RNA duplex, such as, e.g., a Benner's base Z (and/or analogues) or 8-oxo-adenosine (8-oxo-A). As used herein, the term “Benner's base Z” refers to the pyrimidine analogue 6-amino-5-nitro-3-(1′-B-D-2′-deoxyribofuranosyl)-2 (1H)-pyridone (dZ). In one embodiment, a modification includes the introduction of nucleobase analogues or simple heterocycles that boost editing. As used herein, and as commonly understood by the skilled person in the art, the expression “derivative thereof” refers to a derivative of a (modified) nucleobase, nucleoside or nucleotide. For example, a derivative may be a corresponding nucleobase, nucleoside or nucleotide that has been chemically derived from said nucleobase, nucleoside or nucleotide. For instance, a derivative of deoxycytidine may include fluoro-modified deoxycytidine, 5-methyl-2′-deoxycytidine (m5C), or ribocytidine.

As used herein, the term “internucleoside linkage” refers to a linkage between adjacent nucleosides. “Internucleoside linkage” and “linkage” may be used interchangeably. Linkages may be continuous or consecutive. Linkages may be discontinuous or interrupted. As used herein, the term “discontinuous” or “interrupted” means that there are not more than, e.g., 4, 5, 6, 7 or more consecutive internucleoside linkage modifications of the same modification. In some embodiments, the naturally occurring PO linkages are replaced by modified internucleoside linkages. Hence, in some embodiments, the linkage is a non-natural internucleoside linkage. In some embodiments, internucleoside linkage(s) include, but are not limited to phosphorothioate (PS), 3′-methylenephosphonate, 5′-methylenephosphonate, 3′-phosphoroamidate, 2′-5′-phosphodiester, and phosphoryl guanidine (PN) linkages. In another embodiment, the internucleoside linkage modification is a 3′-3′ or 5′-5′ phosphate ester bonds (3′-P-3′ and 5′-P-5′). The internucleoside linkage may be stereopure or stereorandom. Thus, within a particular oligonucleotide, internucleoside linkages may comprise stereopure and stereorandom linkages. In one embodiment, the natural 3′-5′ phosphodiester linkage is replaced by modified internucleoside linkages. In some embodiments, the naturally occurring one or more PO linkages are replaced by modified internucleoside linkages in order to introduce one or more PS linkages or non-phosphorus derived internucleoside linkages.

The term “linker”, as used herein, refers to a bond (e.g., covalent bond), chemical group, or a molecule linking two molecules or moieties, e.g., two domains of a fusion protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20-30, 40-50, 50-60, 60-70, 70-80, 80-90, 90-100, 100-150, or 150-200 amino acids in length. Longer or shorter linkers are also contemplated herein. In some embodiments, the linker is 3 amino acids.

The term “mutation”, as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

As used herein, the term “beneficial editing” refers to the editing of an RNA derived from a wildtype allele (not a mutated allele) to, e.g., modulate the function of a wildtype protein in a useful way to prevent or treat a disease.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

The term “guide RNA” (gRNA) or “guide oligonucleotide”, as used herein refers to a nucleic acid designed to be substantially complementary to the “target sequence”. The guide RNA or guide (antisense) oligonucleotide can comprise endogenous and/or exogenous sequences. Guides can be used in vitro and in vivo. The terms “target RNA sequence”, “target nucleic acid”, “target sequence”, and “target site” are used interchangeably herein to refer to a polynucleotide (nucleic acid, gene, chromosome, genome, etc.) to which a guide RNA sequence is designed to have complementarity. In some embodiments, the guide RNAs described herein comprise two components: a recruitment domain and an antisense domain. The terms “antisense domain” and “antisense sequence” are used interchangeably herein. The antisense domain (i.e., antisense sequence) of the gRNA (or oligonucleotide of the invention) binds to the target RNA. The recruitment domain enables the interaction with the ADAR protein. In some embodiments, the guide RNAs contains three components, a recruitment domain, an antisense domain and two or three recruitment sequences that enhance binding to target that are connected to each other and the antisense domain by a three-nucleotide linker AAA.

The target sequence and guide RNA sequence need not exhibit complete complementarity, provided that there is sufficient complementarity to cause hybridization. Suitable gRNA: RNA binding conditions include physiological conditions normally present in a cell. Other suitable binding conditions (e.g., conditions in a cell-free system) are known in the art; see, e.g., Sambrook, referenced herein and incorporated by reference in its entirety.

The target RNA sequence may be a gene product. The term “gene product”, as used herein, refers to any biochemical product resulting from expression of a gene. Gene products may be RNA or protein. As used herein, the term “target RNA” typically refers to an RNA, which is subject to the editing reaction, and “targeted” by the respective antisense oligonucleotides of the invention. According to the invention, the “target RNA” is a transcript of the Irrk2 gene or any RNA sequence encoding the LRRK2 protein.

A “vector” or “expression vector” is a replicon, such as plasmid, phage, virus, or cosmid, to which another DNA segment, e.g., an “insert,” may be attached or incorporated so as to bring about the replication of the attached segment in a cell. For example, the “insert” may be a construct as described herein. For example, the “insert” may be a construct comprising a target sequence and a guide RNA sequence as described herein.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified”, “mutant”, or “polymorphic” refers to a gene or gene product that displays modifications in sequence and or functional properties (e.g., altered characteristics) when compared to the wild-type gene or gene product. It is noted that naturally-occurring mutants can be isolated; these are identified by the fact that they have altered characteristics when compared to the wild-type gene or gene product.

As used herein, the terms “disease” or “disorder” are used interchangeably to refer to a condition in a subject. In certain embodiments, the condition is a disease associated with the lrrk2 gene in a subject, the severity of which is decreased by inducing an immune response in the subject through the administration of a pharmaceutical composition. According to the invention, the disease, disorder or condition is associated with a mutation in the Irrk2 gene and/or RNA encoding the LRRK2 polypeptide.

As used herein, the term “effective amount” in the context of administering a therapy to a subject refers to the amount of a therapy which has a prophylactic and/or therapeutic effect(s).

As used herein, the terms “prevent”, “preventing” and “prevention” in the context of the present invention and the administration of a therapy(ies) to a subject refers to the inhibition of the development or onset of a disease or a symptom thereof. In one embodiment, it relates to the administration of the compound to a patient who is known to have an increased risk of developing a certain condition, disorder, or disease.

As used herein, the terms “treat”, “treatment”, and “treating” refer in the context of the present invention to the administration of the compound to a patient, which has already developed signs and/or symptoms of a certain condition, disorder, or disease. Beneficial or desired clinical results include, but are not limited to, alleviation of symptoms; diminishment of the extent of a condition, disorder, or disease stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

The terms “subject” or “patient” are used interchangeably and relate to an animal (e.g., mammals) that may need administration of the compound of the invention in the field of human or veterinary medicine. In specific embodiments, the subject is a human subject. The subject may be administered the oligonucleotide of the invention for beneficial editing. The subject may be administered the oligonucleotide of the invention for compensatory editing.

As used herein, the term “pharmaceutically acceptable” means approved by a regulatory agency. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. The formulation should suit the mode of administration.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used.

### 2. Antisense Oligonucleotide

Provided herein are antisense oligonucleotides. In some embodiments, the antisense oligonucleotides comprise a sequence complementary or partially complementary to an RNA target sequence. The ASOs of the invention can be used for several purposes. Advantageously, oligonucleotides provided herein might be useful in the editing of one or more G-to-A mutations.

The inventors of the present invention have discovered that mismatches and/or wobbles (caused by nucleotides in the antisense oligonucleotide that do not form perfect base pairs with the target RNA according to the Watson-Crick base pairing rules) are generally tolerated and improve editing activity of the target RNA sequence and off-target effects. Specifically, it was found that introducing specific mismatches at particular sites between the antisense oligonucleotide sequence and the target RNA sequence enhances off-target effects.

In one aspect provided herein is an antisense oligonucleotide suitable for editing an adenosine in a target RNA nucleic acid sequence to inosine (A-to-I editing), wherein the antisense oligonucleotide comprises a sequence that is substantially complementary to the target RNA nucleic acid sequence: 5′-A C U A C A G C A U U G C-3′ (SEQ ID NO: 1), or complementary to a sequence that has at least 80% sequence identity to SEQ ID NO: 1, wherein A is the target adenosine to be edited in the target nucleic acid; and the antisense oligonucleotide can be represented by: 3′-X₋₅ X₋₄ X₋₃ X₋₂ X₋₁ X₀ X₊₁ X₊₂ X₊₃ X₊₄ X₊₅ X₊₆ X₊₇-5′, wherein X represents a nucleic acid, X₀ represents cytosine (C), deoxycytosine (dC), or uracil (U) directly opposite to the target adenosine to be edited, and wherein there is a mismatch between the antisense oligonucleotide and the target nucleic acid at one or more of the following position(s): a) −5 (X₋₅); b)+3 (X₊₃); c)+7 (X₊₇); d)+7 (X₊₇) and +2 (X₊₂); e)+7 (X₊₇) and −5 (X₋₅); or f)+6 (X₊₆) and −5 (X₋₅). In one embodiment, the oligonucleotide comprises a sequence that is substantially complementary to the target RNA nucleic acid sequence of SEQ ID NO: 2, optionally wherein there is a mismatch between the antisense oligonucleotide and the target nucleic acid at one or more of the following position(s): g)+6 (X₊₆) and −5 (X₋₅); h)+7 (X₊₇) and −10 (X₋₁₀); or i)+3 (X₊₃), −5 (X₋₅), and −8 (X₋₈).

In another aspect provided herein is an antisense oligonucleotide suitable for editing an adenosine in a target RNA nucleic acid sequence to inosine (A-to-I editing), wherein the antisense oligonucleotide comprises a sequence that is substantially complementary to the target RNA nucleic acid sequence: 5′-U G C U G A C U A C A G C A U U G C-3′ (SEQ ID NO: 2), or complementary to a sequence that has at least 80% sequence identity to SEQ ID NO: 2, wherein A is the target adenosine to be edited in the target nucleic acid; and the antisense oligonucleotide can be represented by: 3′-X₋₁₀ X₋₉ X₋₈ X₋₇ X₋₆ X₋₅ X₋₄ X₋₃ X₋₂ X₋₁ X₀ X₊₁ X₊₂ X₊₃ X₊₄ X₊₅ X₊₆ X₊₇-5′, wherein X represents a nucleic acid, X₀ represents cytosine (C), deoxycytosine (dC), or uracil (U) directly opposite to the target adenosine to be edited, and wherein there is a mismatch between the antisense oligonucleotide and the target nucleic acid at one or more of the following position(s): g)+6 (X₊₆) and −5 (X₋₅); h)+7 (X₊₇) and −10 (X₋₁₀); or i)+3 (X₊₃), −5 (X₋₅), and −8 (X₋₈).

The target sequence is selected based upon the gene of interest, i.e., the gene for which site-directed RNA editing is desired. According to the invention, the target RNA sequence comprises a sequence expressed from leucine-rich repeat kinase 2 (lrrk2) gene having a G2019S mutation.

In some embodiments, the target sequence corresponds to a sequence from the Irrk2 gene encoding LRRK2 having a G2019S mutation. In some embodiments, the target sequence comprises the nucleotide sequence 5′-ACUACAGCAUUGC-3′ (SEQ ID NO: 1). In some embodiments, target sequence comprises the nucleotide sequence 5′-UGCUGACUACAGCAUUGC-3′ (SEQ ID NO: 2). In one embodiment, the target RNA nucleic acid sequence comprises a nucleotide sequence having at least 85%, at least 90%, at least 91%, at least 92%, at least 93%, at least 94%, at least 95%, at least 96%, at least 97%, at least 98%, at least 99%, or 100% identity to SEQ ID NO: 1. In one embodiment, the target RNA nucleic acid sequence comprises a nucleotide sequence having at least 85%, at least 90%, at least 91%, at least 92%, at least 93%, at least 94%, at least 95%, at least 96%, at least 97%, at least 98%, at least 99%, or 100% identity to SEQ ID NO: 2.

The antisense oligonucleotide of the invention comprises one or more wobbles (wobble base pairing), bulges, or mismatches, etc. relative to the target sequence. In one embodiment, the mismatch is a paired C-A, C-C, A-C, G-G, C-U or G-U mismatch.

In one embodiment, one or more nucleotide that is not complementary to the nucleotide(s) in the corresponding location in the target nucleic acid is a) cytosine (−5C) at position −5 (X₋₅); b) cytosine (+3C) at position +3 (X₊₃); c) cytosine (+7C) at position +7 (X₊₇); d) cytosine at position +7 (X₊₇) and adenosine at position +2 (X₊₂) is (+7C and +2A); e) cytosine at position +7 (X₊₇) and cytosine at position −5 (X₋₅)(+7C and −5C); f) guanosine at position +6 (X₊₆) and cytosine at position −5 (X₋₅)(+6G and −5C); g) cytosine at position +7 (X₊₇) and guanosine at position −10 (X₋₁₀) (+7C and −10G); h) adenosine at position +3 (X₊₃), cytosine at position −5 (X₋₅), and adenosine at position −8 (X₋₈)(+3A, −5C, and −8A); or i) guanosine at position +6 (X₊₆), cytosine at position +4 (X₊₄), and guanosine at position −10 (X₋₁₀)(+6G, +4C and −10G).

In one embodiment, the one or more nucleotide that is not complementary to the nucleotide(s) in the corresponding location in the target nucleic acid is cytosine (−5C) at position-5 (X₋₅). In one embodiment, the one or more nucleotide that is not complementary to the nucleotide(s) in the corresponding location in the target nucleic acid is cytosine (+3C) at position +3 (X₊₃). In one embodiment, the one or more nucleotide that is not complementary to the nucleotide(s) in the corresponding location in the target nucleic acid is cytosine (+7C) at position +7 (X₊₇). In one embodiment, the one or more nucleotide that is not complementary to the nucleotide(s) in the corresponding location in the target nucleic acid is cytosine at position +7 (X₊₇) and adenosine at position +2 (X₊₂) is (+7C and +2A). In one embodiment, the one or more nucleotide that is not complementary to the nucleotide(s) in the corresponding location in the target nucleic acid is cytosine at position +7 (X₊₇) and cytosine at position −5 (X₋₅)(+7C and −5C). In one embodiment, the one or more nucleotide that is not complementary to the nucleotide(s) in the corresponding location in the target nucleic acid is guanosine at position +6 (X₊₆) and cytosine at position −5 (X₋₅)(+6G and −5C). In one embodiment, the one or more nucleotide that is not complementary to the nucleotide(s) in the corresponding location in the target nucleic acid is cytosine at position +7 (X₊₇) and guanosine at position −10 (X₋₁₀)(+7C and −10G). In one embodiment, the one or more nucleotide that is not complementary to the nucleotide(s) in the corresponding location in the target nucleic acid is adenosine at position +3 (X₊₃), cytosine at position −5 (X₋₅), and adenosine at position −8 (X₋₈)(+3A,-5C, and −8A). In one embodiment, the one or more nucleotide that is not complementary to the nucleotide(s) in the corresponding location in the target nucleic acid is guanosine at position +6 (X₊₆), cytosine at position +4 (X₊₄), and guanosine at position −10 (X₋₁₀)(+6G, +4C and −10G).

In another embodiment, the antisense oligonucleotide comprises 1, 2, 3, 4 or 5 mismatches when aligned with the target nucleic acid. In one embodiment, the ASOs comprise a wobble base pairing. In one embodiment, one or more mismatches are independently a wobble base paring. In one embodiment, the ASOs comprise 2 mismatches or wobble bases. In one embodiment, the ASOs comprise a total of 2 mismatches. In one embodiment, the ASOs comprise 3 mismatches or wobble bases. In one embodiment, the ASOs comprise a total of 3 mismatches. In one embodiment, the ASOs comprise 4 mismatches or wobble bases. In one embodiment, the ASOs comprise a total of 4 mismatches. For instance, the antisense oligonucleotide of the invention may comprise one or more mismatches relative to the RNA target sequence, wherein the one or more mismatches is based on or located at positions: a)+7C, and −10G; b)+3A, −5C, and −8A; c)+6G, and −5C; d)+7C, and −5C; e)+6G, +4C, and −10G; or f)+7C, and +2A. Accordingly, in one embodiment, the antisense oligonucleotide comprises mismatches at positions +7C and −10G. In one embodiment, the antisense oligonucleotide comprises mismatches at positions +3A, −5C, and −8A. In one embodiment, the antisense oligonucleotide comprises mismatches at positions +6G and −5C. In one embodiment, the antisense oligonucleotide comprises mismatches at positions +7C and −5C. In one embodiment, the antisense oligonucleotide comprises mismatches at positions +6G, +4C and −10G. In one embodiment, the antisense oligonucleotide comprises mismatches at positions +7C and +2A.

In one embodiment, the antisense oligonucleotide comprises the general core sequence selected from the list of: a) 3′-X1-X2-X3-X₄-X5-C-X7-X8-X9-X10-C-X11-X12-X13-X14-X15-X16-X17-5′; b) 3′-X1-X2-X3-X₄-X5-X6-X7-X8-X9-X10-C-X11-X12-C-X14-X15-X16-X17-5′; c) 3′-X1-X2-X3-X₄-X5-X6-X7-X8-X9-X10-C-X11-X12-X13-X14-X15-X16-C-5′; d) 3′-X1-X2-X3-X₄-X5-X6-X7-X8-X9-X10-C-X11-A-X13-X14-X15-X16-C-5′; e) 3′-G-X2-X3-X₄-X5-X6-X7-X8-X9-X10-C-X11-X12-X13-C-X15-C-X17-5′; f) 3′-X1-X2-X3-X₄-X5-C-X7-X8-X9-X10-C-X11-X12-X13-X14-X15-X16-C-5′; g) 3′-X1-X2-X3-X₄-X5-C-X7-X8-X9-X10-C-X11-X12-X13-X14-X15-G-X17-5′; or h) 3′-G-X2-X3-X₄-X5-X6-X7-X8-X9-X10-C-X11-X12-X13-X14-X15-X16-C-5′, wherein G, C, and A correspond to guanosine, cytosine and adenosine respectively; and wherein C corresponds to the central nucleotide (X₀) directly opposite the target adenosine (A) to be edited when the antisense domain is hybridised to the target nucleic acid.

In one embodiment, the antisense oligonucleotide comprises at least 85%, or at least 90% sequence complementarity to the target nucleic acid sequence.

To reduce the amount of (or avoid any) off-target editing and enhance on-target editing levels, oligonucleotides of the invention may comprise a specific antisense sequence (see, Table 1). That is, antisense oligonucleotides may comprise an optimized sequence as listed in Table 1. Specifically, an antisense oligonucleotide may comprise or be based on a sequence selected from the group consisting of SEQ ID NO: 3 to SEQ ID NO: 102. Oligonucleotides may comprise a combination of one of more of said sequences.

The antisense sequences as disclosed herein (Table 1) may differ in their on-target editing levels (%) and/or off-target editing levels (%).

The on-target editing level (%) at position 0 may vary between the oligonucleotides of the invention. In one embodiment, the on-target editing level (%) at position 0 is above 70%. In one embodiment, the on-target editing level (%) at position 0 is above 65%. In one embodiment, the on-target editing level (%) at position 0 is above 60%. In one embodiment, the on-target editing level (%) at position 0 is above 50%. In one embodiment, the on-target editing level (%) at position 0 is from 60% to 75%.

In some cases, this leads to a off-target editing level that is 0 (zero). Accordingly, in one embodiment, the oligonucleotides of the invention show no off-target editing. This means that in some embodiment, the off-target editing level is 0. In one embodiment, the off-target editing level (%) is 0 (zero) at position −5. In one embodiment, the off-target editing level (%) is 0 (zero) at position −2. In one embodiment, the off-target editing level (%) is 0 (zero) at position +3. In one embodiment, the off-target editing level (%) is 0 (zero) at position −5 and at position +3. In one embodiment, the off-target editing level (%) is 0 (zero) at position −5, −2, and +3.

In one embodiment, the antisense oligonucleotide comprises a sequence selected from the group consisting of: a) SEQ NO: 8, b) SEQ NO: 21, c) SEQ NO: 34, d) SEQ NO: 40, e) SEQ NO: 42, and f) SEQ NO: 43. In one embodiment, the antisense oligonucleotide comprises SEQ ID NO: 8. In one embodiment, the antisense oligonucleotide comprises SEQ ID NO: 21. In one embodiment, the antisense oligonucleotide comprises SEQ ID NO: 34. In one embodiment, the antisense oligonucleotide comprises SEQ ID NO: 40. In one embodiment, the antisense oligonucleotide comprises SEQ ID NO: 42. In one embodiment, the antisense oligonucleotide comprises SEQ ID NO: 43. In one embodiment, the antisense oligonucleotide comprises SEQ ID NO: 48. In one embodiment, the antisense oligonucleotide comprises SEQ ID NO: 49.

Table 2 shows examples of optimized antisense sequences for editing LRRK2 G2019. In some embodiments, oligonucleotides of the invention comprise a sequence shown in Table 2.

The antisense oligonucleotide of the invention may be sequence optimized in that it does not contain certain nucleotides at certain positions. For instance, there may not be a guanosine in the oligonucleotide at position −7 (X₋₇). Hence, in one embodiment, the oligonucleotide does not comprise a guanosine at position −7 (X₋₇). In one embodiment, the oligonucleotide does not comprise a guanosine at position −2 (X₋₂). In one embodiment, the oligonucleotide does not comprise a cytosine at position −1 (X₋₁). In one embodiment, the oligonucleotide does not comprise a thymidine at position −1 (X₋₁). In one embodiment, the oligonucleotide does not comprise an adenosine at position 0 (X₀). In one embodiment, the oligonucleotide does not comprise a guanosine at position 0 (X₀). In one embodiment, the oligonucleotide does not comprise an adenosine at position +1 (X₊₁). In one embodiment, the oligonucleotide does not comprise one or more of the following mismatches selected from the group consisting of: −7G, −2G, −1C, −1T, 0A, 0G, and +1A, or any combination thereof.

The antisense oligonucleotides of the invention may have different lengths to provide optimal RNA editing. The shorter the oligonucleotide, the better might be the endosomal escape. Moreover, toxicity of the particular oligonucleotide may also depend on its length. Also, shorter oligonucleotides may experience higher specificity. On the other hand, while longer oligonucleotides may bind stronger or faster to their respective RNA target, editing-boosting bulges, mismatches and wobbles may also work better in long oligonucleotides. As a result, there is a benefit and/or trade-off for both long and short oligonucleotides of the invention. Notably, for an viral vector-encoded guide RNA, the oligonucleotide may have a length of up to 200 nt or even >300 nt including flexible linker and circularization elements. Accordingly, the oligonucleotides of the invention may be of varying lengths. The oligonucleotides may range from about 15-80 nucleotides in length, e.g., 15-45 nucleotides, 25-45 nucleotides or 30-35 nucleotides in length. In one embodiment, the oligonucleotide has a length of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. In one embodiment, the oligonucleotide has a length of more than 80 nucleotides.

In one embodiment, the sequence of the oligonucleotide is 15-80 nucleotides, preferably 25-45 nucleotides, most preferably 30-35 nucleotides in length. In one embodiment, the oligonucleotide has a length of 25-45 nucleotides. In one embodiment, the oligonucleotide has a length of 30-35 nucleotides. In certain embodiments, the oligonucleotide has a length of 15-50 nucleotides. In some embodiments, the oligonucleotide has a length of 15-80, 15-70, 15-60, 15-50, 15-40, 15-33, or 15-38 nucleotides. In some embodiments, the oligonucleotide has a length of at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In one embodiment, the oligonucleotide has a length of 15-60, 15-55, 15-50, 15-45, 15-40, 15-35, 15-30, 25-45, or 30-35 nucleotides. In one embodiment, the nucleic acid sequence of the oligonucleotide is 15-30 nucleotides in length. In one preferred embodiment, the nucleic acid sequence of the oligonucleotide is 15-25 nucleotides in length. In one embodiment, the nucleic acid sequence of the oligonucleotide is 18 nucleotides in length.

The antisense oligonucleotides of the invention may have a nucleotide sequence that is shorter than the sequence of SEQ ID NO: 2. In one embodiment, the oligonucleotide sequence comprises SEQ ID NO: 1. In one embodiment, the oligonucleotide sequence comprises 10, 11, 12, 13, 14, 15, 16, or 17 nucleotides. In one embodiment, the oligonucleotide sequence has a length of at least 15 or exactly 15 nucleotides. In one embodiment, the oligonucleotide has a length of 13 nucleotides. In one embodiment, the oligonucleotide has a length of 14 nucleotides. In one embodiment, the oligonucleotide contains 7 nt 3′ to X₀. In one embodiment, the oligonucleotide contains 6 nt 3′ to X₀. In one embodiment, the oligonucleotide contains 5 nt 3′ to X₀. In one embodiment, the oligonucleotide contains 4 nt 3′ to X₀. In one embodiment, the oligonucleotide contains 8 nt 3′ to X₀. In one embodiment, the oligonucleotide contains 9 nt 3′ to X₀. In one embodiment, the oligonucleotide contains 10 nt 3′ to X₀. In one embodiment, the oligonucleotide contains 7 nt 5′ to X₀. In one embodiment, the oligonucleotide contains 6 nt 5′ to X₀. In one embodiment, the oligonucleotide contains 5 nt 5′ to X₀. In one embodiment, the oligonucleotide contains 4 nt 5′ to X₀. In one embodiment, the oligonucleotide contains 3 nt 5′ to X₀.

In one embodiment, the oligonucleotide has a length of at least 13, 14, 15, 16, 17, or 18 nucleotides and no more than 4 nucleotides are deoxynucleotides. In one embodiment, the oligonucleotide has a length of 15 nucleotides and comprises internucleoside linkage modifications and/or 2′-sugar modifications. Ranges and lengths intermediate to the above recited ranges and lengths are also contemplated to be part of the invention.

The oligonucleotide of the invention may contain three components: a recruitment domain, an antisense domain and two or three recruitment sequences that enhance binding to target that are connected to each other and the antisense domain by a three-nucleotide linker AAA. Hence, in one embodiment, the oligonucleotide comprises at least 1 recruitment sequence (RS). In one embodiment, the oligonucleotide comprises 1 recruitment sequence. In one embodiment, the oligonucleotide comprises 2 recruitment sequences. In one embodiment, the oligonucleotide comprises 3 recruitment sequences. In one embodiment, the oligonucleotide comprises 1-3 recruitment sequences. In one embodiment, the oligonucleotide comprises 1, 2, 3, 4, 5, 6 or more recruitment sequences. In one embodiment, the oligonucleotide comprises 1, 2 or 3 recruitment sequences.

The oligonucleotides of the invention may comprise one or more recruitment sequences or combinations thereof. For instance, the oligonucleotide of the invention comprises a first, second and third recruitment sequence. In one embodiment, a first recruitment sequence is 3′-UGAAAAGCAGCACAU-5′ (SEQ ID NO: 107), a second recruitment sequence is 3′-UAUCUCAAACCAUCA-5′ (SEQ ID NO: 106), and a third recruitment sequence is 3′-AAGCAGGCGAUCCAAGGAAC-5′ (SEQ ID NO: 105). In another embodiment, a first recruitment sequence is 3′-GUGUGAAAAGCAGCACAUUG-5′ (SEQ ID NO: 110), a second recruitment sequence is 3′-GGUAUCUCAAACCAUCAGCU-5′ (SEQ ID NO: 109), and a third recruitment sequence is 3′-GUGUGAAAAGCAGCACAUUG-5′ (SEQ ID NO: 108). In another embodiment, a first recruitment sequence is 3′-AGCAGGCGAUCCAAGGAACC-5′ (SEQ ID NO: 113), a second recruitment sequence is 3′-AACUCCAUCACCAACAUCCG-5′ (SEQ ID NO: 112), and a third recruitment sequence is 3′-GUCAGCCAAAAUCAAGUCAG-5′ (SEQ ID NO: 111). In one embodiment, a first recruitment sequence is 3′-UCUCAAACCAUCAGC-5′ (SEQ ID NO: 116), a second recruitment sequence is 3′-CGAUCCAAGGAACCC-5′ (SEQ ID NO: 115), and a third recruitment sequence is 3′-CAUCCGGGGACGAAU-5′ (SEQ ID NO: 114). In some embodiment, the oligonucleotides of the invention comprise any combination of first, second and third recruitment sequences listed in Table 3. In one embodiment, the oligonucleotide comprises gRNA1, gRNA2, gRNA3, or gRNA4 of Table 3.

As used herein, the target sequence comprises a sequence encoding leucine-rich repeat kinase 2 (LRRK2). In one embodiment, the antisense oligonucleotide does not comprise a target sequence. In one embodiment, the target RNA nucleic acid encodes a LRRK2 polypeptide comprising a mutation corresponding to a G2019S mutation.

Compared to reference oligonucleotides or compositions, the provided oligonucleotides or compositions are surprisingly effective in target editing and reducing off-target effects. In some embodiments, a change is measured by an increase of a desired mRNA and/or protein level compared to a reference sample or condition. In some embodiments, a change is measured by an increase in the editing efficacy (%) mediated by the oligonucleotide or composition of the invention. In some embodiments, a change is measured by an increase in stability of the oligonucleotide or composition comprising the same. In some embodiments, a change is measured in the levels of cytotoxicity, viability, apoptosis or immune activation. In some embodiments, a change is detected by means of luminescence and/or gene expression. In some embodiments, toxicity and therapeutic efficacy can be determined by standard pharmaceutical procedures in cell cultures or experimental animals, e.g., for determining the LD₅₀ (the dose lethal to 50% of the population) and the LD₅₀ (the dose therapeutically effective in 50% of the population). In some embodiments, data obtained from the cell culture assays or animal studies can be used in formulating a range of dosages for use in humans.

The oligonucleotides of the invention may be modified to enhance cellular uptake and/or used in combination with various delivery systems. For instance, the oligonucleotides of the invention may be modified at their 5′ and/or 3′ termini to enhance cellular uptake of the oligonucleotide. The oligonucleotides of the invention may comprise one or more different linkers, tags or coupling agents at either one or both termini. For example, the oligonucleotides may comprise amino-linkers, preferably C6-amino-linkers. Hence, in some embodiments, the oligonucleotides of the invention comprise a C6-amino-linker at the 5′ terminus. In some embodiments, the oligonucleotides comprise a C6-amino-linker at the 3′ terminus. The oligonucleotides may comprise a moiety, which enhances cellular uptake of the oligonucleotide, e.g., N-acetylgalactosamine (GalNAc). Hence, in some embodiments, the chemically modified oligonucleotide comprises a moiety or is conjugated to a moiety that enhances cellular uptake of the oligonucleotide. Preferably, the moiety enhancing cellular uptake is a triantennary N-acetyl galactosamine (GalNAc3), which is preferably conjugated to the 3′ terminus or to the 5′ terminus of the oligonucleotide. Further, the oligonucleotides of the invention may be delivered using nanoparticles, liposomes, antibody-conjugated liposomes, cationic lipids, polymers, cell-penetrating peptides, or microparticles well known in the art. Alternatively, a plasmid can be used to deliver the oligonucleotide ‘as is’ (also referred to as ‘naked’).

Furthermore, the oligonucleotides of the invention may be chemically modified. That is, the oligonucleotides may comprise a chemically modified backbone. Accordingly, in one embodiment, the antisense oligonucleotide comprises at least one chemical modification. While not intending to be bound by any particular theory of operation, it is believed that nucleobase and backbone linkage modifications of antisense oligonucleotides are useful in stabilizing and improving the editing efficacy and lysosomal stability of the oligonucleotides of the invention. Moreover, these modifications also have the potential to reduce the off-target editing of the different ASOs. Since the one or more modifications can be synthetically transferred to various oligonucleotide sequences, such modifications have the potential to improve the editing efficacy of oligonucleotides with different specificities, e.g., oligonucleotides targeting transcripts of the Irrk2 gene and/or RNA encoding LRRK2.

The ASOs of the invention can be modified as shown in in WO2022/253810, which is herewith incorporated by reference in its entirety. Also, the ASOs of the invention may be chemically modified as shown in WO2023/099494 (incorporated herewith by reference in its entirety). In some instances, the ASOs of the invention may comprise a mixture of different modifications at the 2′-position of the sugar residue, and no more than 6 nucleotides with the same 2′-modification. Also, avoiding uniform blocks of more than 6 nucleotides with the same 2′-modification prevented a strong loss of editing activity with natural ADARs.

The oligonucleotides of the invention comprise a central base triple (CBT) comprising a core sequence of 3′-X₋₁ X₀ X₊₁-′5, wherein X₀ is the central nucleotide directly opposite to the target adenosine in the target RNA. The CBT may carry different modifications and permutations of the various modifications. In certain embodiments, the CBT is chemically modified. That is, positions X₋₁, X₀ and/or X₊₁ may carry modifications at the 2′ position. For example, only one position within the CBT may be chemically modified or two positions within the CBT may be chemically modified. In other cases, all positions within the CBT may be chemically modified. At least two of the three nucleotides of the CBT may be chemically modified at the 2′ position of the sugar moiety, may be deoxyribonucleosides, or a combination thereof. For example, the X₋₂ nucleotide may carry a 2′-O-alkyl-modification and the X₋₃ nucleotide may carry a 2′-fluoro (2′-F)-modification. Each of the three nucleosides of the CBT may either be singularly or a combination of: (a) a deoxyribonucleotide; and/or (b) 2′-FANA-modification; and/or (c) 2′-O-methyl-modification; and/or (d) 2′-F-modification. In one embodiment, (i) X₊₁ is 2′-F, 2′-FANA, DNA, or 2′-O-methyl; and/or (ii) X₀ is 2′-FANA or DNA; and/or (iii) X₋₁ is 2′-FANA, DNA, or 2′-O-methyl. The 2′-O-alkyl-modification may be a 2′-O-methyl(2′-OMe)-modification. In some embodiments, position X-1 is 2′-Fluoro-RNA, 2′-FANA or DNA. In some embodiments, position X₀ is 2′-FANA or DNA. In some embodiments, position X₊₁ is 2′-FANA or DNA. CBT modification may comprise any permutation of the modifications described above.

In one embodiment, at least 10% of nucleotides of the oligonucleotide are 2′-F-modified and at least 10% of nucleotides of the oligonucleotide are 2′-O-alkyl-modified, wherein no more than 6 consecutive nucleotides have the same 2′-modification. In one embodiment, the internucleoside linkage modification content is at least 15%. In another embodiment, the regions 3′ and 5′ to the CBT do not contain more than a total of 6 deoxyribonucleosides and the internucleoside linkage modification content is at least 30%. In one embodiment, the regions 3′ and 5′ to the CBT have a total deoxyribonucleoside content of 5-50%. In one embodiment, 20-100% of nucleotides of the oligonucleotide are 2′-modified, preferably wherein 50-100% of nucleotides are 2′-modified nucleotides.

The oligonucleotides may benefit from having a base level of internucleoside linkage modifications. For example, the oligonucleotides may have at least 15% of their internucleoside modified to achieve good RNA editing. For example, no more than 95%, 90%, 85%, 80%, 70%, 60%, 50%, 40%, 30% or 20% of the linkages are internucleoside linkage modifications; or 15-90% of the linkages are internucleoside linkage modifications, preferably wherein 40-80%, most preferably 45-60%, of the linkages are internucleoside linkage modifications.

In one embodiment, the oligonucleotide comprises at least one internucleoside linkage modification selected from the group consisting of phosphorothioate (PS), 3′-methylenephosphonate, 5′-methylenephosphonate, 3′-phosphoroamidate, 2′-5′phosphodiester, and phosphoryl guanidine (PN), preferably wherein the at least one internucleoside linkage modification is PS. In one embodiment, the antisense oligonucleotide comprises at least 60% of PS linkages or 60%-95% PS linkages. In some embodiments, the oligonucleotide comprises at least two internucleoside linkage modifications. In some embodiments, the oligonucleotide comprises at least three internucleoside linkage modifications.

Also, it is beneficial for the oligonucleotides to incorporate modifications at the 2′-position of the nucleotides and that such modification should be composed of different groups. For example, it may be beneficial for the oligonucleotide to have 20-70% of nucleotides 2′-Fluoro (F)-modified, preferably 35-65% of nucleotides 2′-F-modified; and/or 20-60% of nucleotides 2′-O-methyl(2′-OMe)-modified, preferably 25-55% of nucleotides 2′-OMe-modified. In some cases, the 2′-O-alkyl-modification is a 2′-O-methyl(2′-OMe)-modification.

In one embodiment, (i) no more than 4, 5, or 6 consecutive nucleotides are 2′-F-modified; and/or (ii) no more than 4, 5, or 6 consecutive nucleotides are 2′-OMe-modified. In one embodiment, less than 6, 5, 4, or 3 consecutive nucleotides have the same 2′-modification. In one embodiment, the X₋₂ nucleotide carries a 2′-O-alkyl-modification, optionally wherein the 2′-O-alkyl-modification is a 2′-O-methyl(2′-OMe)-modification; and/or wherein the X-3 nucleotide carries a 2′-fluoro (2′-F)-modification.

Loop-hairpin structured oligonucleotides have previously been described (WO 2020/001793) and used successfully to harness ADARs with chemically modified oligonucleotides. However, they are comparably large and—without being bound by any theory—the inventors believe that a more intelligent design of the ASO can form a substrate duplex that is also very well and quickly recognized by endogenous ADAR so that the large recruitment motifs can be omitted.

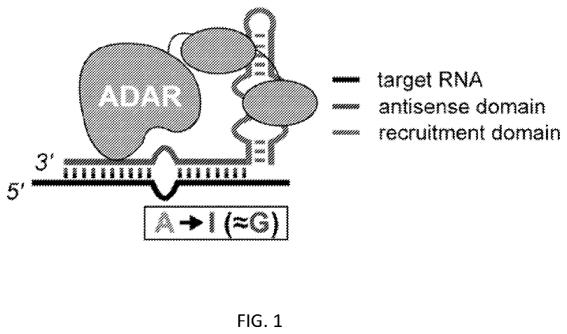

In some embodiments, the oligonucleotide does not comprise a secondary structure. In some embodiments, the oligonucleotide does not comprise a hairpin-loop structure. In some embodiments, the oligonucleotide does not comprise a hairpin-loop structured ADAR recruitment motif or domain. In some embodiments, the oligonucleotides does not comprise a recruitment domain. The oligonucleotides of the invention may only contain an antisense domain that binds to the target RNA (FIG. 1). In one embodiment, the oligonucleotides comprise only an antisense domain.

### 3. Fusion Constructs

Described herein are fusion constructs. In some embodiments, provided herein are fusion constructs comprising a guide RNA sequence and a target sequence. The fusion constructs provided herein find use in various methods, including methods of high-throughput screening for selecting guide RNAs for use in site-directed RNA editing.

In some embodiments, the fusion construct possesses a stem loop secondary structure.

In some embodiments, the fusion construct comprises a target sequence. The target sequence in the fusion constructs is selected based upon the gene of interest (i.e., the gene for which site-directed RNA editing is desired). In some embodiments, the target sequence in the fusion constructs comprises a mutated sequence. For example, the target sequence may comprise a nucleotide sequence possessing one or more mutations, wherein said one or more mutations result in a disease phenotype. In some embodiments, the target sequence in the fusion constructs comprises a sequence from leucine-rich repeat kinase 2 (LRRK2) having a G2019S mutation. The G2019S mutation is a G to A point mutation at the nucleotide level. In some embodiments, the target sequence in the fusion constructs comprises a sequence from the kinase domain of LRRK2 having a G2019S mutation. In some embodiments, the target sequence comprises the nucleotide sequence ACUACAGCAUUGC (SEQ ID NO: 1). In some embodiments, the target sequence comprises the nucleotide sequence UGCUGACUACAGCAUUGC (SEQ ID NO: 2). In some embodiments, the target sequence in the fusion constructs comprises a nucleotide sequence having at least 80% sequence identity (e.g., at least 80%, at least 85%, at least 90%, at least 91%, at least 92%, at least 93%, at least 94%, at least 95%, at least 96%, at least 97%, at least 98%, at least 99%, or 100% identity) to SEQ ID NO: 1. In some embodiments, the target sequence in the fusion constructs comprises a nucleotide sequence having at least 80% sequence identity (e.g., at least 80%, at least 85%, at least 90%, at least 91%, at least 92%, at least 93%, at least 94%, at least 95%, at least 96%, at least 97%, at least 98%, at least 99%, or 100% identity) to SEQ ID NO: 2. In some embodiments, the guide RNA sequence comprises an antisense domain. The antisense domain of the gRNA binds to the target RNA. Accordingly, selection of the sequence of the antisense domain depends on the sequence of the target RNA of interest (i.e., the desired RNA to be edited). The antisense domain may comprise any suitable number of nucleotides. In some embodiments, the antisense domain comprises 10-50 nucleotides. For example, in some embodiments the antisense domain comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In some embodiments, the antisense domain comprises more than 50 nucleotides. In some embodiments, the antisense domain comprises 10-30 nucleotides. In some embodiments, the antisense domain comprises 15-25 nucleotides. In some embodiments, the antisense domain comprises 18 nucleotides. In some embodiments, the length of the antisense domain depends on whether the guide RNA additionally comprises a recruitment domain. For example, guide RNA sequences lacking a recruitment domain may contain antisense domains of longer length compared to guide RNA sequences containing both a recruitment domain and an antisense domain. In some embodiments, the antisense domain comprises one or more mutations compared to the prototype antisense domain sequence 3′-ACGACTGATGCCGTAACG-5′ (SEQ ID NO: 6). In some embodiments, the antisense domain comprises a sequence having at least 80% identity (e.g., at least 80%, at least 85%, at least 90%, at least 91%, at least 92%, at least 93%, at least 94%, at least 95%, at least 96%, at least 97%, at least 98%, at least 99%, or 100% identity) to SEQ ID NO: 6. In some embodiments, the antisense domain comprises a sequence shown in Table 1. In some embodiments, the antisense domain comprises a sequence shown in Table 2. In some embodiments, the antisense domain comprises SEQ ID NO: 8, SEQ ID NO: 21, SEQ ID NO: 34, SEQ ID NO: 40, SEQ ID NO: 42, or SEQ ID NO: 43.

In some embodiments, the guide RNA described herein lacks a recruitment domain. For example, in some embodiments the guide RNA comprises a target sequence encoding for LRRK2 and an antisense domain and does not comprise a recruitment domain. In some embodiments, the target sequence and the antisense domain in the fusion constructs are linked by a loop structure, such that the construct forms a stem-loop secondary structure. The loop structure in the fusion constructs may comprise any suitable number of nucleotides. In some embodiments, the loop structure in the fusion constructs comprises 3-50 nucleotides. In some embodiments, the loop structure comprises 3-50 nucleotides, 3-45 nucleotides, 3-40 nucleotides, 3-35 nucleotides, 3-30 nucleotides, 3-25 nucleotides, 3-20 nucleotides, 3-15 nucleotides, 3-10 nucleotides, or 3-7 nucleotides. In some embodiments, the loop structure is a pentaloop (i.e., comprises 5 nucleotides). Suitable loop structures are described in International Application Publication No. WO2022087272A1, the entire contents of which are incorporated herein by reference for all purposes.

In some embodiments, the guide RNA comprises an antisense domain and a recruitment domain. The guide RNA sequence may be optimized for RNA editing, such as by making one or more mutations in the antisense domain and/or recruitment domain as described herein.

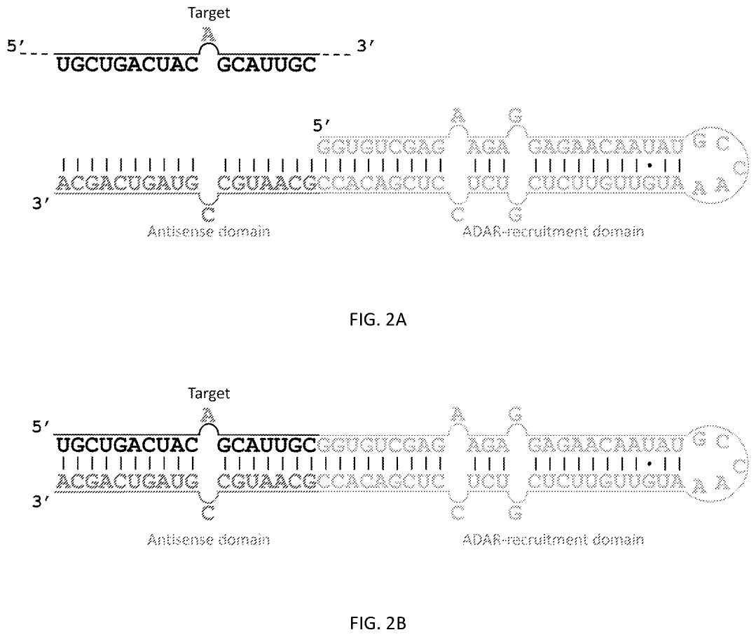

In some embodiments, the antisense domain in the fusion constructs is substantially complementary to the target sequence. Accordingly, nucleotides within the antisense domain base pair with corresponding nucleotides on the target sequence in the fusion constructs, thus forming the secondary structure of the construct. The base pairing need not be 100%. For example, one or more nucleotides in the antisense domain do not base pair with the nucleotide in the corresponding location in the target sequence. In some embodiments, the antisense domain comprises one or more mutations that disrupt perfect complementarity (i.e., disrupt base pairing). For example, the antisense domain may comprise one or more mutations that disrupt base pairing with the target sequence in the fusion constructs. In some embodiments, the antisense domain comprises one or more mutations, such that there are one or more mismatches between the antisense domain and the target sequence in the fusion construct. In some embodiments, the antisense domain comprises at least one, at least two, at least three, at least four, at least five, at least six, or more than six mismatches when hybridized to the target sequence of SEQ ID NO: 1 in the fusion construct. In some embodiments, the antisense domain comprises at least one, at least two, at least three, at least four, at least five, at least six, or more than six mismatches when hybridized to the target sequence of SEQ ID NO: 2 in the fusion construct. Such mismatches are identified in Table 1, which contains exemplary antisense sequences that can be used in the fusion constructs described herein. Mismatched positions are identified in Table 1 relative to the C to A mismatch shown in red in FIG. 2B.

In some embodiments, the guide RNA sequence comprises a recruitment domain. The recruitment domain (also referred to herein as the ADAR-recruiting part), facilitates the interaction with the ADAR or ADAR fusion protein. The recruitment domain is configured to bind (i.e., recruit) one or more ADAR proteins or fusions thereof. For example, the recruitment domain may be configured to recruit an ADAR1, an ADAR2 protein or a fusion thereof. In some embodiments, the recruitment domain recruits at least an ADAR2 protein. The recruitment domain may comprise any suitable number of nucleotides. For example, the recruitment domain may comprise 15-100 nucleotides. In some embodiments, the recruitment domain comprises about 15, about 20, about 25, about 30, about 35, about 40, about 45, about 50, about 55, about 60, about 65, about 70, about 75, about 80, about 85, about 90, about 95, or about 100 nucleotides.

In some embodiments, the recruitment domain is based upon the sequence of an endogenous (i.e., naturally occurring) ADAR target. The recruitment domain may possess one or more modifications compared to the endogenous ADAR target, which may enhance ADAR recruitment or interactions. For example, the recruitment domain may be based upon the sequence of the GRIA2 R/G site, an endogenous target for ADAR2.

In some embodiments, the recruitment domain comprises a first strand (i.e., a 5′ strand) and a second strand (i.e., a 3′ strand) connected by a loop structure (also referred to herein as a loop sequence). The first strand and the second strand exhibit complementary base pairing, thus assisting in the formation of the stem loop structure of the construct. In some embodiments, this base pairing is disrupted by one or more mutations within the first strand and/or the second strand of the recruitment domain. In some embodiments, an unmodified recruitment domain refers to a recruitment domain that exhibits base pairing with no disruptions (i.e., perfect complementarity), whereas a mutated recruitment domain refers to a domain comprising one or more mutations in the first strand or the second strand that disrupt base pairing. In other words, an unmodified recruitment domain comprises a first strand with perfect complementarity to a second strand, whereas a mutated recruitment domain comprises a first strand and a second strand with substantial (i.e., at least 60%), but not perfect complementarity.

In some embodiments the recruitment domain comprises a first strand and a second strand connected by a loop structure. The loop structure may comprise any suitable number of nucleotides. In some embodiments, the loop structure comprises 3-50 nucleotides. In some embodiments, the loop structure comprises 3-50 nucleotides, 3-45 nucleotides, 3-40 nucleotides, 3-35 nucleotides, 3-30 nucleotides, 3-25 nucleotides, 3-20 nucleotides, 3-15 nucleotides, 3-10 nucleotides, or 3-7 nucleotides. In some embodiments, the loop structure is a pentaloop structure.

In some embodiments, the recruitment domain comprises the sequence: GGUGUCGAGAAGAGGAGAACAAUAUGCCAAAUGUUGUUCUCGUCUCCUCGACAC C (SEQ ID NO: 103). In some embodiments, the recruitment domain comprises a sequence having at least 80% sequence identity (e.g., at least 80%, at least 85%, at least 90%, at least 91%, at least 92%, at least 93%, at least 94%, at least 95%, at least 96%, at least 97%, at least 98%, at least 99%, or 100% identity) to SEQ ID NO: 103. In some embodiments, the recruitment domain comprises a sequence having at least 80% sequence identity (e.g., at least 80%, at least 85%, at least 90%, at least 91%, at least 92%, at least 93%, at least 94%, at least 95%, at least 96%, at least 97%, at least 98%, at least 99%, or 100% identity) to SEQ ID NO: 103, and the pentaloop structure of the recruitment domain comprises GCCAA (SEQ ID NO: 104). An exemplary recruitment domain is shown in FIG. 2A and FIG. 2B. Additional suitable recruitment domains and/or pentaloop sequences for the recruitment domain may be used, including those described in WO2022087272A1, the entire contents of which are incorporated herein by reference for all purposes.

In some embodiments, the fusion construct comprises a combination of mutations. The combination of mutations may be in one or more regions within the construct. For example, the fusion construct may comprise multiple mutations in the guide RNA. For example, the construct may comprise one or more mutations within the antisense domain (i.e., one or more mutations that disrupt a given base pairing with a corresponding nucleotide in the target sequence) of the guide RNA and one or more mutations within the recruitment domain of the guide RNA (i.e., one or more mutations that disrupt or restore base pairing between the first strand and the second strand of the recruitment domain).

In some embodiments, the fusion construct comprises one or more components in addition to the guide RNA sequence and the target sequence. For example, in some embodiments the fusion construct comprises at least one recruitment sequence that enhances binding to the target sequence. In some embodiments, the fusion construct comprises at least two recruitment sequences. In some embodiments, the at least two recruitment sequences are connected to each other and to the antisense domain by a linker. In some embodiments, the fusion construct comprises at least three recruitment sequences. In some embodiments, the fusion construct comprises at least three recruitment sequences that are connected to each other and the antisense domain by a linker. In some embodiments, the linker comprises 3-5 amino acids. In some embodiments, the linker comprises AAA. In some embodiments, the fusion construct comprises at least one recruitment sequence shown in Table 3. In some embodiments, the fusion construct comprises at least two recruitment sequences shown in Table 3. In some embodiments, the fusion construct comprises three recruitment sequences shown in Table 3. In some embodiments, the fusion construct comprises the combination of recruitment sequences shown in Table 3 for any one of gRNA1, gRNA2, gRNA3, or gRNA4. In some embodiments, the antisense domain comprises a sequence shown in Table 2. In some embodiments, the antisense domain comprises SEQ ID NO: 8, SEQ ID NO: 21, SEQ ID NO: 34, SEQ ID NO: 40, SEQ ID NO: 42, or SEQ ID NO: 43, and the fusion construct further comprises a combination of recruitment sequences shown in Table 3 for any one of gRNA1, gRNA2, gRNA3, or gRNA4.

For example, the fusion construct may additionally comprise one or more components to facilitate determination of whether the construct is effectively expressed in a cell of interest. For example, the fusion construct may additionally comprise sequences encoding a fluorescent protein, which enables visualization of whether a construct is expressed in a cell of interest. In some embodiments, the fusion construct comprises intervening sequences between the guide RNA sequence and the target sequence. Such intervening sequences may comprise any suitable number of nucleic acids. For example, the fusion construct may comprise a sequence encoding a fluorescent protein, which may assist in determining that the construct is expressed in a cell of interest.

### 4. Compositions and Uses Thereof

The antisense oligonucleotides (or ASOs) provided herein may be incorporated into compositions of the invention. For instance, targeted delivery of oligonucleotides to liver hepatocytes using bi- or triantennery N-acetylgalactosamine (GalNAc) conjugates has previously described for, e.g., treating liver diseases, including Hepatitis B virus (HBV), non-alcoholic Fatty Liver Disease and genetic diseases (Debacker et al., 2020). Accordingly, provided herein are pharmaceutical compositions comprising an antisense oligonucleotide of the invention or a pharmaceutically acceptable salt thereof.

The present disclosure provides oligonucleotide compositions of antisense oligonucleotides described herein. In some embodiments, the compositions are pharmaceutical compositions. As used herein, pharmaceutical composition means a mixture of substances suitable for administering to an individual. For example, a pharmaceutical composition may comprise one or more active pharmaceutical agents (such as an antisense oligonucleotide) and a sterile aqueous solution. In one embodiment, the composition contains one or more oligonucleotides of the invention. In one embodiment, provided herein is a pharmaceutical composition or a pharmaceutically acceptable salt thereof comprising an antisense oligonucleotide of the invention, or a vector of the invention.

The pharmaceutical compositions provided herein can be in any form that allows for the composition to be administered to a subject. The compositions may be used in methods of treating and/or preventing a genetic disorder, condition, or disease. In a specific embodiment, the pharmaceutical compositions are suitable for veterinary and/or human administration. In some embodiments, an oligonucleotide or composition containing an oligonucleotide described herein is administered to a human. In some embodiments, the human subject to be administered an oligonucleotide or composition containing an oligonucleotide is any individual at risk of developing a disease or disorder associated with a G-to-A mutation in the Irrk2 gene. In one embodiment, the patient suffers from a disease or disorder associated with a G-to-A mutation in the Irrk2 gene.

In one embodiment, a composition comprises an oligonucleotide of the invention in an admixture with a pharmaceutically acceptable carrier. In some embodiments, the pharmaceutically acceptable carrier can simply be a saline solution. This can be isotonic or hypotonic.

In some embodiments, a pharmaceutical composition may comprise one or more other therapies in addition to an oligonucleotide of the invention.

In certain embodiments, the compositions of the invention further include diluents of various buffer content (e.g., Tris-HCl, acetate, phosphate), pH, and ionic strength, and additives such as detergents and solubilizing agents (e.g., Tween 80, Polysorbate 80), anti-oxidants (e.g., ascorbic acid, sodium metabisulfite), preservatives (e.g., Thimersol, benzyl alcohol), and bulking substances (e.g., lactose, mannitol). In some embodiments, the material may be incorporated into particulate preparations of polymeric compounds such as polylactic acid, polyglycolic acid, etc. or into liposomes. In some embodiments, hyaluronic acid may also be used. Such compositions may influence the physical state, stability, rate of in vivo release, and/or rate of in vivo clearance of the present ASOs and derivatives. In some embodiments, the compositions may be prepared in liquid form, or may be in dried powder, such as lyophilized form.

In certain embodiments, the pharmaceutical compositions described herein additionally comprise one or more salts, e.g., sodium chloride, calcium chloride, sodium phosphate, monosodium glutamate, and aluminium salts (e.g., aluminium hydroxide, aluminium phosphate, alum (potassium aluminium sulfate), or a mixture of such aluminium salts). In other embodiments, the pharmaceutical compositions described herein do not comprise salts.

The pharmaceutical compositions described herein can be included in a container, pack, or dispenser together with instructions for administration.

The invention generally describes the use of the antisense oligonucleotide and/or composition comprising the same in the medical setting. Specifically, the antisense oligonucleotides of the invention are for site-directed editing of a target RNA, i.e., binding to the target RNA via the targeting sequence and recruiting a deaminase). The present invention describes antisense oligonucleotide and/or composition for use in the treatment or prevention of a genetic disorder, condition, or disease as well as methods for treating or preventing a genetic disorder, condition, or disease. Site-directed editing may take place in vitro, in vivo or ex vivo.

The oligonucleotide or composition comprising the same may be used in the treatment and/or prevention of a medical condition. In one aspect provided herein is the use of an oligonucleotide of the invention and/or a composition comprising the same in the treatment or prevention of a genetic disorder, condition, or disease.

In one embodiment, the genetic disorder, condition, or disease is associated with a G-to-A mutation. In one embodiment, the genetic disorder, condition, or disease is associated with a G-to-A mutation in the Irrk2 gene. In one embodiment, the genetic disorder, condition, or disease is associated with a change in amino acid sequence in the LRRK2 protein.

In one embodiment, the genetic disorder, condition, or disease is Parkinson's disease (PD).

In one embodiment, the pharmaceutical compositions provided herein are used for the treatment of a LRRK2-associated disease or condition. In one embodiment, the pharmaceutical compositions provided herein are used for the treatment of Parkinson's Disease (PD).

The oligonucleotide of the invention or the (pharmaceutical) composition may be administered, for example, orally in any orally acceptable dosage form including, but not limited to, capsules, tablets, aqueous suspensions, or solutions, or parenterally. In some embodiments, formulations suitable for parenteral administration comprise sterile aqueous preparations of at least one embodiment of the present disclosure, which are approximately isotonic with the blood of the intended recipient. The amount of oligonucleotide or composition to be administered, the dosage and the dosing regimen can vary from cell type to cell type, the disease to be treated, the target population, the mode of administration (e.g., systemic versus local), the severity of disease and the acceptable level of side activity. In some embodiments, the amount of oligonucleotides administered in a pharmaceutical composition is dependent on the subject being treated, the subject's weight, the manner of administration, and/or disease to be treated.

Various delivery systems can be used to deliver the oligonucleotides of the invention. An oligonucleotide according to the invention can be delivered as is (i.e., naked and/or in isolated form) to an individual, an organ (the eye), or specifically to a cell. When administering an oligonucleotide according to the invention, it is preferred that the oligonucleotide is dissolved in a solution that is compatible with the delivery method. Such delivery may be in vivo, in vitro or ex vivo. Nanoparticles and micro-particles that may be used for in vivo ASO delivery are well known in the art. Alternatively, a plasmid can be provided by transfection using known transfection reagents. In a preferred embodiment, the oligonucleotides of the present invention are administered and delivered ‘as is’, also referred to as ‘naked’. Nevertheless, the art contains multiple ways of delivering oligonucleotides to cells, either in vitro, ex vivo or in vivo. That is, depending on the disease, disorder or infection that needs to be treated, or on the cell, tissue or part of the body that needs to be reached by the oligonucleotides of the present invention (e.g., in case of beneficial editing), an administration route or delivery method may be selected. Examples for delivery when an oligonucleotide is not delivered naked, are delivery agents or vehicles such as nanoparticles, like polymeric nanoparticles, liposomes, antibody-conjugated liposomes, cationic lipids, polymers, or cell-penetrating peptides.

Delivery of the oligonucleotides may be performed indirectly by administering one or more vectors that encode and direct the expression of the oligonucleotide. Hence, provided herein is a vector that comprises the engineered polynucleotide of the invention. In one embodiment, the vector is a viral vector or non-viral vector. In one embodiment, the viral vector is a lentiviral vector, retroviral vector, adenovirus vector, adeno-associated virus vector, or alphavirus vector. In one embodiment, the non-viral vector is a plasmid, cosmid, or phage.

Combinations of in vitro and in vivo methods of contacting a cell are also possible. Contacting a cell may be direct or indirect. Contacting of a cell with an oligonucleotide may be done in vitro or in vivo. Known methods can be adapted for use with an oligonucleotide of the invention (see, e.g., Akhtar et al. (1992) and WO1994/02595, which are incorporated herein by reference in their entireties).

The oligonucleotides of the invention of compositions comprising the same may be administered to various groups of subjects or patients. In certain embodiments, the patient is in need of treatment.

In certain embodiments, an oligonucleotide or composition containing an oligonucleotide described herein is administered to a naive subject, i.e., a subject that does not have a disease or disorder. In one embodiment, an oligonucleotide or composition containing an oligonucleotide is administered to a subject. In one embodiment, an oligonucleotide or composition provided herein is administered to a naive subject that is at risk of developing a disease or disorder.

In certain embodiments, an oligonucleotide or a composition containing an oligonucleotide of the invention is administered to a patient who has been diagnosed with a disease or disorder. In some embodiments, an oligonucleotide or a composition containing an oligonucleotide of the invention is administered to a patient before symptoms manifest or symptoms become severe.

Also provided herein is a method of treating a subject suffering from a genetic disorder, condition, or disease, wherein the method comprises administering to the subject in need thereof an effective amount of an antisense oligonucleotide of the invention, a vector of the invention or pharmaceutically acceptable salt thereof, or a pharmaceutical composition of the invention. In one embodiment, the genetic disorder, condition, or disease is Parkinson's Disease (PD), Lewy body dementia, frontotemporal dementia, corticobasal 3 dementia, progressive supranuclear palsy, Alzheimer's disease, tauopathy disease, or alpha-4 synucleinopathy. In one embodiment, the genetic disorder, condition, or disease is a LRRK2-associated disease or condition. In one embodiment, the LRRK2-associated disease or condition is associated with a LRRK2 G2019S mutation.

In one embodiment, the subject is a human subject.

The oligonucleotides, vectors or compositions (comprising said oligonucleotides) may be used for prophylactic and/or therapeutic uses. Hence, also provided herein are antisense oligonucleotides of the invention, vector of the invention and/or pharmaceutical compositions of the invention for use in therapy. In one embodiment, the antisense oligonucleotide, the vector, or the pharmaceutical composition is for use in the treatment of a disease or disorder, where in the disease or disorder is selected form the group consisting of Parkinson's Disease (PD), Lewy body dementia, frontotemporal dementia, corticobasal 3 dementia, progressive supranuclear palsy, Alzheimer's disease, tauopathy disease, or alpha-4 synucleinopathy. In one embodiment, the disease or disorder comprises the Irrk2 gene or transcript thereof. In one embodiment, the disease or disorder comprises the LRRK2 protein.

### 5. High-Throughput Screening Methods

ite-directed A-to-I RNA editing presents the opportunity to temporarily manipulate essential biological processes, such as cell signaling or inflammation, whose permanent alteration would otherwise have serious consequences, and allows for the precise regulation of the biological outcome due to the tunability of introducing a change in RNA (e.g. potentially from 0% to 100%). Inosine is biochemically interpreted as guanosine by the cellular machinery, as such A-to-I editing formally introduces A-to-G point mutations in RNA, which offers the opportunity to manipulate or restore genetic information. Adenosine deaminases acting on RNA (ADARs) naturally catalyze A-to-I editing at millions of sites within double-stranded RNA (dsRNA) regions of the transcriptome of higher organisms and play important roles in the regulation of protein function, RNA splicing, immunity and RNA interference.

In some embodiments, provided herein are systems and methods that find use to identify, select, produce, and utilize gRNAs that maximize the RNA editing yield. In some embodiments, provided herein is a high-throughput screening method for selecting guide RNAs for use in site-directed RNA editing. In some embodiments, the method comprises generating a plurality of fusion constructs as described herein. The fusion constructs comprise a target sequence and a guide RNA sequence as described herein. In some embodiments, the target sequence is derived from the LRRK2 gene with a G2019S point mutation.

The methods further comprise inducing expression of the fusion construct in a suitable cell. For example, the method may further comprise transfecting cells expressing adenosine deaminases acting on RNA (ADARs) or cells expressing ADAR fusion proteins with the fusion constructs. The method further comprises determining whether a fusion construct effectively induces one or more mutations in nucleic acid isolated from the cells relative to a control. Any suitable cells expressing ADARs or ADAR fusion proteins may be used. Suitable cells include eukaryotic cells including but not limited to yeast cells, higher plant cells, animal cells, insect cells, and mammalian cells. Non-limiting examples of eukaryotic cells include simian, bovine, porcine, murine, rat, avian, reptilian and human cells.

Transfection methods may be assisted by the use of suitable cell permeabilizing agents (e.g., lipofectamine) or may be performed by other suitable techniques such as electroporation. The fusion constructs may be housed in a suitable vector prior to delivery to the cell. Suitable vectors include viral vectors (e.g., lentiviral vectors, retroviral vectors, adenovirus vectors, adeno-associated virus vectors, alphavirus vectors, etc.) and non-viral vectors (e.g., plasmids, cosmids, phages, etc.). Following achieving the desired expression of the construct within the cell, the method further comprises determining whether a given fusion construct effectively induces one or more modifications in nucleic acid isolated from the cells relative to a control. Accordingly, in some embodiments the method further comprises isolating nucleic acid from the cells. The isolated nucleic acid may be RNA.

In some embodiments, determining whether a fusion construct induces one or more modifications in nucleic acid isolated from the population of cells expressing the fusion construct comprises sequencing the isolated nucleic acid. In some embodiments, the one or more modifications in nucleic acid isolated from the population of cells comprises a correction of the mutation (e.g., G2019S mutation) initially present in the target sequence. For example, RNA may be isolated from the cells and sequencing may be performed to determine whether the G2019S mutation initially present in the target sequence has been corrected.

In some embodiments, the method further comprises determining whether expression of the construct effectively induced a modification in the RNA compared to a control. For example, the method may comprise determining the sequence of the isolated nucleic acid (e.g., RNA). A variety of suitable sequencing methods and technologies may be used to determine the sequence of the nucleic acid strands. For example, the sequencing method may be Sanger sequencing. As another example, the sequencing method may be a next generation sequencing technology (e.g., next generation RNA sequencing technology). The term next generation sequencing, or “NGS”, refers to a variety of sequencing techniques that permit simultaneous sequencing of millions of nucleic acid sequences, and is otherwise referred to as high-throughput sequencing or massively parallel sequencing. In some embodiments, RNA may be isolated from the cells and cDNA of the target RNA/gRNA fusions may be prepared for subsequent sequencing with NGS (such as by using a platform commercially available from Illumina). For the sequencing library preparation, NGS adapters with different indexes may be used, which allows the concurrent analysis of multiple constructs. To analyze the sequencing data, a computational pipeline may be used which enables the detection of editing levels within the target RNA sequences and the identification of the corresponding gRNAs.

In some embodiments, the methods described herein may be used to identify gRNAs comprising one or more optimized features such that a guide RNA comprising the optimized feature(s) effectively induces site-directed RNA editing. The optimized features may be selected from the antisense domain, the recruitment domain, the loop sequence, and the recruitment sequence(s). For example, the methods described herein may be used to identify optimized antisense domains, target sequences, loop sequences, recruitment domain sequences, and/or recruitment sequences. In some embodiments, the methods described herein may be used to identify optimized antisense domains. Accordingly, such optimized antisense domains may be used in circular guide RNAs or in guide RNAs lacking a recruitment domain. For example, optimized antisense domains may be used in circular guide RNAs or in guide RNAs lacking a recruitment domain for methods of site-directed gene editing. Alternatively, optimized antisense domains may be used in combination with another optimized feature in a guide RNA, such as an optimized recruitment domain and/or an optimized loop sequence. In some embodiments, the methods described herein may be used to identify gRNAs containing an optimized recruitment domain. For example, the methods may identify gRNAs containing optimized first strand sequences and/or optimized second strand sequences for a recruitment domain. In some embodiments, the methods may identify optimized loop sequences. In some embodiments, the methods may identify optimized recruitment sequences, which may be optimized in combination with one or more additional features (e.g., antisense sequences, for example) to generate a guide RNA. Accordingly, the methods described herein may be used to assist in the generation of guide RNAs containing one or more optimized features, including an optimized antisense domain, an optimized target sequence, and optimized loop sequence, optimized recruitment sequences, and/or an optimized recruitment domain sequence.

### 6. Guide RNAs and Therapeutic Methods

ite-directed RNA editing finds use to reverse the disease phenotypes caused by point mutations without the safety concerns, which are associated with genome engineering, and requiring only administration of the gRNA as a therapeutic.

In some embodiments, provided herein are methods for harnessing endogenous ADARs for the correction of the G2019S mutation in the LLRK2 gene causing Parkinson's Disease. Subsequently to identifying optimized gRNA(s) as described herein, said gRNA(s) may be used in the methods for treating Parkinson's Disease.

Generally described herein are methods for site-directed RNA editing. According to the invention, provided herein is a method for site-directed A-to-I editing of a target RNA, comprising providing to a cell or subject an oligonucleotide according to the invention, a vector of the invention or a pharmaceutical composition according to the invention. The methods comprise selecting a gRNA by a method/platform as described herein and providing a construct comprising the guide RNA to a cell or a subject. In some embodiments, the guide RNA is a gRNA as described herein.

Also provided herein is an in vitro method for deaminating at least one specific adenosine present in a target RNA sequence in a cell, wherein the method comprises the steps of: (a) contacting the target nucleic acid with an antisense oligonucleotide of the invention, a vector of the invention or a pharmaceutical composition of the invention; (b) allowing uptake by the cell of the antisense oligonucleotide; (c) allowing annealing of the antisense domain to the target RNA sequence; and (d) allowing a mammalian ADAR enzyme comprising a natural dsRNA binding domain as found in the wild-type enzyme to deaminate the target adenosine in the target RNA sequence to an inosine. In some embodiment, the cell endogenously expresses ADAR. In one embodiment, the ADAR is a human ADAR. In one embodiment, the ADAR is human ADAR1.

The oligonucleotides of the invention may comprise a guideRNA. In some embodiments, are guide RNAs for use in site-directed RNA editing. The guide RNA may be any suitable guide RNA described herein. The guide RNA may be identified using a high-throughput screening method as described herein. In some embodiments, the guide RNA comprises an antisense domain that is substantially complementary or perfectly complementary to a portion of the LRRK2 gene sequence comprising a G2019S mutation. In some embodiments, the guide RNA comprises an antisense domain that is substantially complementary or perfectly complementary to the target sequence SEQ ID NO: 1 or SEQ ID NO: 2. In some embodiments, the guide RNA comprises an antisense domain shown in Table 1. In some embodiments, the antisense domain comprises a sequence shown in Table 2. In some embodiments, the antisense domain comprises SEQ ID NO: 8, SEQ ID NO: 21, SEQ ID NO: 34, SEQ ID NO: 40, SEQ ID NO: 42, or SEQ ID NO: 43. In some embodiments, the guide RNA sequence comprises a recruitment domain. In some embodiments, the guide RNA sequence comprises the recruitment domain of SEQ ID NO: 103. In some embodiments, the guide RNA sequence further comprises at least one recruitment sequence. In some embodiments, the guide RNA sequence further comprises at least one recruitment sequence shown in Table 3.

In some embodiments, the guide RNA or construct comprising the same may be formulated into a composition for delivery to the cell or subject. For example, the construct may be formulated into a composition for parenteral administration. The term “parenteral” refers to any suitable non-oral route of administration, including subcutaneous, intramuscular, intravenous, intrathecal, intracisternal, intraarterial, intraspinal, intraepidural, intradermal, and the like. The construct may be formulated with any suitable excipients, stabilizers, preservatives, and the like. In some embodiments, the composition may be provided to a subject suffering from Parkinson's Disease. Accordingly, in some embodiments provided herein are methods for treating Parkinson's disease, comprising providing to a subject in need thereof a composition comprising a gRNA as described herein (i.e., an optimized gRNA). The gRNA may be identified using a high-throughput screening method as described herein.

It is understood that endogenous ADARs and/or engineered ADAR fusions may be suitable for use in the methods for site-directed RNA editing described herein. For example, the guide RNAs (including optimized guide RNAs) identified by a screening method described herein may be well suited for use with ADAR fusion proteins in the methods described herein.

All references, including publications, patent applications, and patents, cited herein are hereby incorporated by reference to the same extent as if each reference were individually and specifically indicated to be incorporated by reference and were set forth in its entirety herein.

Preferred embodiments are described herein, including the best mode known to the inventors for carrying out the methods described herein. Variations of those preferred embodiments may become apparent to those of ordinary skill in the art upon reading the foregoing description. The inventors expect skilled artisans to employ such variations as appropriate, and the inventors intend for the methods to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### EXAMPLES

The examples shown in the following are merely illustrative and shall describe the present invention in a further way. These examples shall not be construed to limit the present invention thereto.

### Example 1

Adenosine deaminases acting on RNA, or ADAR proteins, ubiquitously expressed in human cells, bind double-stranded RNA and edit adenosine to inosine, which is biochemically interpreted as guanosine. Antisense oligonucleotides or guide RNAs can be used to recruit endogenous ADARs for site-directed RNA editing. Targeting of several endogenous transcripts with these guide RNAs yielded RNA editing efficiencies of up to 80-90% in various cell types. Site-directed editing is highly specific and maintains the natural editing homeostasis (Merkle et al, 2019). Importantly, this new technology holds several advantages over alternative genome engineering techniques like CRISPR by (1) harnessing endogenous ADAR proteins, which are already abundantly expressed throughout human tissues, thus eliminating the need of Cas9 delivery and associated toxicity; (2) editing patients' RNA, which, unlike DNA editing, allows for reversible and therefore safer manipulation; (3) exhibiting very high specificity. Accordingly, this approach offers a precise and efficient platform for transcriptome engineering, including the repair of pathogenic G-to-A mutations, without the significant safety risks associated with other genome engineering approaches.

ADAR-recruiting gRNAs may contain two functional domains; the first, antisense domain binds to the target RNA, while the second, ADAR-recruiting domain, binds and recruits the endogenous ADAR protein. A schematic of an ADAR-recruiting gRNA is shown in FIG. 1. The oligonucleotides of the invention may only contain an antisense domain that binds to the target RNA (FIG. 1). gRNAs can be chemically modified to improve the target affinity, in vivo stability, and cellular uptake (Levin, 2019; Merkle et al, 2019). However, the efficiency of site-directed ADAR editing continues to lag behind the up-to 100% editing efficiencies seen in some natural ADAR substrates (Nishikura, 2016). Therefore, what is needed are high-throughput screens to optimize ADAR gRNA designs, along with effective LRRK2 G2019S gRNAs.

Deviations from perfect double-stranded RNA (dsRNA) may enhance the editing efficiency and specificity. Without wishing to be found by theory, it is possible that deviations from perfect dsRNA may enhance editing efficiency and/or specificity by defining the productive ADAR binding registers along the RNA duplex and creating unfavorable contexts for ‘off-target’ A residues. In addition, only a handful of recruitment domain variants have been tested, despite the diversity of structured RNA contexts in natural ADAR substrates. These observations suggest that there is ample room for the development of ADAR guide RNAs with high editing efficiency and specificity.

The potential for LRRK2 G2019S repair by ADAR-recruiting gRNAs can be evaluated in two stages. First, editing levels of G2019S mRNA can be measured after gRNA transfection into FlpIn TRex 293 cells expressing the G2019S mutant and. This can be used to narrow down the number of guide RNAs for subsequent assays.

The LRRK2 G2019S guides can be designed at least in part based on published prototypes that gave the highest editing for other targets (Merkle et al, 2019) and those described in WO2022087272A1, the entire contents of which are incorporated herein by reference for all purposes. High-throughput screening to optimize the LRRK2 guide RNA can also be performed, which will further inform the guide design. The G2019S editing level can be assessed by Sanger sequencing, and the transcriptome-wide off-target editing will be determined by RNA-Seq.

### Example 2

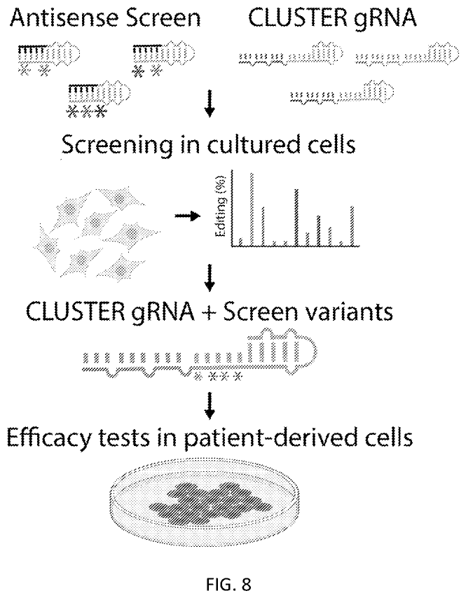

To achieve the highest possible efficiency and specificity of guide RNAs, a workflow that (1) screens a library of >10,000 gRNA sequences for their ability to edit the G-to-A point mutation in LRRK2 G2019S; (2) further improves the top gRNA candidates identified in the screen by addition of CLUSTERs of recruitment sequences for increased target affinity; (3) tests the gRNAs in an in vitro model of human LRRK2 G2019S to determine the editing level, and phenotypic effects, was developed. This workflow can generate improved guide designs for subsequent in-vivo tests. An exemplary workflow is shown in FIG. 8.

A next-generation sequencing approach can be used, including both unbiased investigation of randomized guide sequences and rationally designed gRNA variants. To enable simultaneous assessment of the guide sequence and associated editing level in a high-throughput fashion, a hybrid construct can be used, in which the target region from the LRRK2 G2019S RNA sequence is covalently fused to the guide RNA (FIG. 2B). The resulting hairpin structures simulate the duplex formed between the target RNA and a trans-acting guide RNA, while enabling preservation of the information on the editing level and the guide sequence in the same sequencing construct.

The starting point for library design is based on a prototype comprising a short (~18 nt.) antisense region and a recruitment domain derived from an endogenous ADAR substrate (R/G motif from GluR2 mRNA, or ‘GluR/G’; FIG. 2A).

Given the apparent importance of unpaired residues (located in bulges and loops) for high editing efficiency, the antisense sequence can first be randomized. Hand-mixed bases can be used during library synthesis to ensure comprehensive coverage of single, double, and triple mismatches, while still enabling decent coverage of higher-order mutants. Designed variants containing all possible single-nucleotide insertions and deletions in the antisense strand, as well as structural perturbations observed in natural targets, can also be used.

The recruitment domain variants can include high-efficiency natural ADAR substrates, as well as designed structures, including double recruitment domains, Z-RNA forming motifs (targeting ADAR1's Z-RNA binding domains) and other perturbations. Recruitment domain length, sequence, stability, and distance from the editing site can be systematically varied. In some embodiments, the recruitment domain can be omitted altogether by extending the length of the antisense region. Hairpin variants consisting entirely of the LRRK2 sequence, and its complement can also be used.

The hairpin library, comprising all variants discussed above, can be introduced into a reporter plasmid and delivered into cells expressing or inducibly expressing ADAR proteins. For example, the hairpin library can be introduced into a reporter plasmid and delivered into FlpIn TRex 293 cells expressing endogenous ADARs or inducibly expressing individual ADAR proteins (ADAR1 or ADAR2). Upon extraction of total RNA, the variants can be reverse transcribed using a primer targeting a constant flanking region. The reverse transcription primer can also include a randomized unique molecular identifier (UMI) sequence, to account for PCR and sequencing artifacts in subsequent sequencing analysis (Buenrostro et al, 2014; Kivioja et al, 2012). Sequencing can be performed using the Illumina HiSeq platform.

The most promising sequence and structural features identified in the screen can subsequently be implemented in trans, i.e., by delivering plasmid- or AAV-expressed guides to cells expressing the LRRK2 G2019S sequence. The extent of editing can be assessed by Sanger sequencing and amplicon deep sequencing.

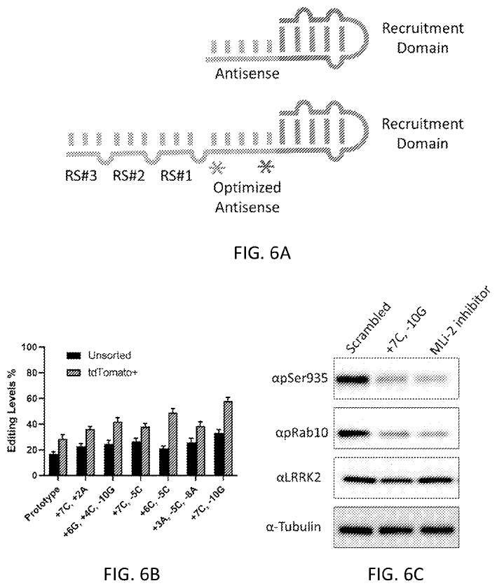

To assess the potency of optimized guides and associated phenotypic effects in a clinically relevant, the human iN model system (Bieri et al, 2019), specifically-neuronal progenitor cells differentiated from PD patient derived iPS cells with the G2019S mutation can be used. Optimized guides can reduce mutant LRRK2 kinase activity, potentially restoring it to physiological levels. This can be assessed by immunolabeling of phosphorylated LRRK2 at Ser935 and the LRRK2 substrate Rab10 (Lis et al, 2018), as shown in FIG. 6B. This phenotype can be used as a functional readout of ability to revert the G2019S mutation, since the deregulation of Rab10 is associated with increased LRRK2 activity and neurodegeneration (Alessi & Sammler, 2018). These provide benchmarks to test the ability of the guide RNA editing approach to suppress a LRRK2 G2019S phenotype. Transcriptome-wide off-target effects can be assessed by RNA-seq (Ramaswami et al, 2013; Tan et al, 2017).

### Example 3

Described herein are guide RNAs that are capable of efficient site-directed RNA editing of a pathogenic G2019S mutation in the LRRK2 kinase, the most common genetic cause of Parkinson's disease. Guide RNAs were identified using a high-throughput screen. Several guide RNAs are provided that can harness the endogenous ADAR deaminase for efficient A-to-G RNA editing of the LRRK2 G2019S target sequence with high efficiency and specificity in cultured cells. Attachment of clusters of recruitment sequences was able to significantly boost editing levels of the identified guide RNAs (Reautschnig et al., 2022). The guide RNAs disclosed herein can reduce pathogenic effects of mutant LRRK2 in cells, including reducing LRRK2 hyperactivation to potentially physiological levels. These guide RNAs are capable of efficiently editing the G2019S mutation in LRRK2 transcripts in pre-clinical models, such as human iPSC-derived neuronal progenitor cells derived from Parkinson's disease patients that carry the mutation on both alleles.

FIG. 2A-2B show a prototype guide RNA design for editing LRRK2 G2019S. As shown, the guide RNA comprises a recruitment domain and an antisense oligonucleotide. The target sequence hybridizes with the antisense portion, as shown in FIG. 2B, to generate a hairpin structure. Optimized guide RNA sequences were developed from this prototype. Suitable variables to modify include, for example, the sequence, length, and/or indels for the antisense sequence, and/or the sequence and structure of the recruitment domain.

### Optimized Guide RNA Antisense Sequences

Table 1. List of top 100 antisense sequences (out of ~10,000 guide RNAs) from the high-throughput screen are shown below, and sequence changes relative to the prototype sequence are indicated. All these sequences enhance on-target editing levels by >1.25 folds above the prototype design (42.2%). For each antisense sequence, the on-target editing levels at the Position 0 adenosine as well as off-target editing levels at other adenosines are indicated. The recruitment domain was kept constant and has a 5′ to 3′ sequence of:

(SEQ ID NO: 103)
GGUGUCGAGAAGAGGAGAACAAUAUGCCAAAUGUUGUUCUCGUCUCCUC
GACACC

TABLE 1

| | Mismatched | | On-target editing | Off-target editing levels (%) | | |
|---|---|---|---|---|---|---|
| SEQ ID | base and | Antisense sequence | levels (%) at | Position | Position | Position |
| NO. | position | (3′ to 5′) | Position 0 | −5 | −2 | +3 |
| SEQ ID | +7U, +3C, −3G | ACGACUGGUGCCGCAACU | 90.91 | 0 | 58.33 | 66.67 |
| NO. 3 | | | | | | |
| SEQ ID | +7U, +4U, +3C, | CCGACUAAUGCCGCUACU | 85.71 | 0 | 36.36 | 9.09 |
| NO. 4 | −4A, −10C | | | | | |
| SEQ ID | +3A, −1U | ACGACUGAUUCCGAAACG | 83.33 | 0 | 33.33 | 0 |
| NO. 5 | | | | | | |
| SEQ ID | +6U, +3C, −9U | AUGACUGAUGCCGCAAUG | 75.86 | 0 | 16.67 | 66.67 |
| NO. 6 | | | | | | |
| SEQ ID | +3C,−10C | CCGACUGAUGCCGCAACG | 73.68 | 0 | 13.79 | 37.93 |
| NO. 7 | | | | | | |
| SEQ ID | +7C, −10G | GCGACUGAUGCCGUAAC | 73.66 | 0 | 0 | 0 |
| NO. 8 | | C | | | | |
| SEQ ID | +6G, +3G, −5C | ACGACCGAUGCCGGAAGG | 72.73 | 0 | 6.67 | 0 |
| NO. 9 | | | | | | |
| SEQ ID | +3C, −6G, −9G | AGGAGUGAUGCCGCAACG | 71.43 | 0 | 7.14 | 50 |
| NO. 10 | | | | | | |
| SEQ ID | +7A, +3C, −6G, | AAGAGUGAUGCCGCAACA | 71.43 | 0 | 9.09 | 54.55 |
| NO. 11 | −9A | | | | | |
| SEQ ID | +2U, −9U, −10C | CUGACUGAUGCCUUAACG | 70 | 0 | 7.14 | 0 |
| NO. 12 | | | | | | |
| SEQ ID | +6U, +4G, −4C | ACGACUCAUGCCGUGAUG | 69.44 | 0 | 10 | 0 |
| NO. 13 | | | | | | |
| SEQ ID | +5C, +4G, −9G | AGGACUGAUGCCGUGCCG | 69.23 | 0 | 7.69 | 0 |
| NO. 14 | | | | | | |
| SEQ ID | +7U, −10G | GCGACUGAUGCCGUAACU | 69.23 | 0 | 7.69 | 2.56 |
| NO. 15 | | | | | | |
| SEQ ID | +7C, +2C | ACGACUGAUGCCCUAACC | 69.23 | 0 | 13.89 | 0 |
| NO. 16 | | | | | | |
| SEQ ID | +7U, +6G, +5G, | ACGACUGAUGCCCUAGGU | 69.23 | 0 | 34.12 | 3.12 |
| NO. 17 | +2C | | | | | |
| SEQ ID | +7U, +6U, +3C | ACGACUGAUGCCGCAAUU | 68.75 | 0 | 19.23 | 26.92 |
| NO. 18 | | | | | | |
| SEQ ID | +7C, +3C, −9U, | GUGACUGAUGCCGCAACC | 68.42 | 0 | 3.13 | 21.88 |
| NO. 19 | −10G | | | | | |
| SEQ ID | +6A, +3C, −10G | GCGACUGAUGCCGCAAAG | 68.42 | 0 | 15.79 | 42.11 |
| NO. 20 | | | | | | |
| SEQ ID | +3A, −5C, −8A | ACAACCGAUGCCGAAACG | 68.41 | 0 | 5.26 | 0 |
| NO. 21 | | | | | | |
| SEQ ID | +7C, +4C, −10C | CCGACUGAUGCCGUCACC | 66.67 | 0 | 22.21 | 0 |
| NO. 22 | | | | | | |
| SEQ ID | +7C, −9A | AAGACUGAUGCCGUAACC | 66.67 | 0 | 23.46 | 0 |
| NO. 23 | | | | | | |
| SEQ ID | +5C, −4A | ACGACUAAUGCCGUACCG | 66.67 | 0 | 29.17 | 8.33 |
| NO. 24 | | | | | | |
| SEQ ID | +7C, +3C, +2U, | ACGCCUGACGCCUCAACC | 66.67 | 0 | 66.67 | 0 |
| NO. 25 | −2C, −7C | | | | | |
| SEQ ID | +5C, +4U, −9U | AUGACUGAUGCCGUUCCG | 65.52 | 0 | 11.11 | 5.56 |
| NO. 26 | | | | | | |
| SEQ ID | +7C, +4U | ACGACUGAUGCCGUUACC | 65.38 | 0 | 0 | 17.24 |
| NO. 27 | | | | | | |
| SEQ ID | +7A, +3G | ACGACUGAUGCCGGAACA | 64.86 | 3.85 | 3.85 | 0 |
| NO. 28 | | | | | | |
| SEQ ID | +7U, −4A | ACGACUAAUGCCGUAACU | 64.29 | 0 | 5.41 | 0 |
| NO. 29 | | | | | | |
| SEQ ID | −5C, −8C, −9G | AGCACCGAUGCCGUAACG | 63.64 | 0 | 9.09 | 0 |
| NO. 30 | | | | | | |
| SEQ ID | +5° C., +4U | ACGACUGAUGCCGUUCCG | 63.64 | 0 | 7.14 | 7.14 |
| NO. 31 | | | | | | |
| SEQ ID | +7C, +5U | ACGACUGAUGCCGUAUCC | 63.64 | 0 | 18.18 | 9.09 |
| NO. 32 | | | | | | |
| SEQ ID | +5G, −8C, −10G | GCCACUGAUGCCGUAGCG | 63.48 | 0 | 18.18 | 18.18 |
| NO. 33 | | | | | | |
| SEQ ID | +6G, −5C | ACGACCGAUGCCGUAAG | 63.47 | 0 | 5.22 | 0 |
| NO. 34 | | G | | | | |
| SEQ ID | +6G, +3C, −8U | ACUACUGAUGCCGCAAGG | 63.16 | 0 | 5.26 | 23.68 |
| NO. 35 | | | | | | |
| SEQ ID | +7U, +3C, −5A | ACGACAGAUGCCGCAACU | 62.69 | 0 | 10.53 | 21.05 |
| NO. 36 | | | | | | |
| SEQ ID | +6A, +4G, −6G, | UCGAGUGAUGCCGUGAAG | 62.51 | 0 | 8.96 | 0 |
| NO. 37 | −10U | | | | | |
| SEQ ID | +7A, −5C | ACGACCGAUGCCGUAACA | 61.54 | 2.08 | 4.17 | 0 |
| NO. 38 | | | | | | |
| SEQ ID | −5C, −9A, −10C | CAGACCGAUGCCGUAACG | 61.54 | 0 | 11.54 | 0 |
| NO. 39 | | | | | | |
| SEQ ID | +7C, −5C | ACGACCGAUGCCGUAACC | 61.42 | 0 | 4.85 | 0 |
| NO. 40 | | | | | | |
| SEQ ID | +5G, +4G, +2U | ACGACUGAUGCCUUGGCG | 61.42 | 21.1 | 0 | 0 |
| NO. 41 | | | | | | |
| SEQ ID | +6G, +4C, −10G | GCGACUGAUGCCGUCAG | 61.21 | 0 | 3.12 | 0 |
| NO. 42 | | G | | | | |
| SEQ ID | +7C, +2A | ACGACUGAUGCCAUAACC | 60.91 | 0 | 5 | 0 |
| NO. 43 | | | | | | |
| SEQ ID | +7A, +6G, +4G, | ACCACUGAUGCCGUGAGA | 60.91 | 0 | 6.55 | 0 |
| NO. 44 | −8C | | | | | |
| SEQ ID | +5G, −5C | ACGACCGAUGCCGUAGCG | 60.91 | 0 | 10.53 | 2.63 |
| NO. 45 | | | | | | |
| SEQ ID | +7C, +3C, −9G | AGGACUGAUGCCGCAACC | 60.91 | 0 | 5 | 25 |
| NO. 46 | | | | | | |
| SEQ ID | +7C, −5C,−10C | CCGACCGAUGCCGUAACC | 60 | 0 | 3.33 | 2.35 |
| NO. 47 | | | | | | |
| SEQ ID | +3C, +1U, −5C | ACGACCGAUGCUGCAACG | 60 | 0 | 6.45 | 0 |
| NO. 48 | | | | | | |
| SEQ ID | +5° C., −3U | ACGACUGUUGCCGUACCG | 59.72 | 0 | 6.45 | 0 |
| NO. 49 | | | | | | |
| SEQ ID | +7U, +3C | ACGACUGAUGCCGCAACU | 59.57 | 0 | 6.94 | 23.61 |
| NO. 50 | | | | | | |
| SEQ ID | +7C, −9G | AGGACUGAUGCCGUAACC | 59.09 | 0 | 8.51 | 0 |
| NO. 51 | | | | | | |
| SEQ ID | +7C, −5C, −9G | AGGACCGAUGCCGUAACC | 58.97 | 0 | 25.81 | 6.45 |
| NO. 52 | | | | | | |
| SEQ ID | +7A, +2C | ACGACUGAUGCCCUAACA | 58.82 | 2.94 | 3.45 | 0 |
| NO. 53 | | | | | | |
| SEQ ID | +7A, −10G | GCGACUGAUGCCGUAACA | 58.82 | 0 | 10.26 | 3.42 |
| NO. 54 | | | | | | |
| SEQ ID | +7A, −10U | UCGACUGAUGCCGUAACA | 58.46 | 0 | 17.65 | 5.88 |
| NO. 55 | | | | | | |
| SEQ ID | +7C, −10C | CCGACUGAUGCCGUAACC | 58.33 | 0 | 10 | 0 |
| NO. 56 | | | | | | |
| SEQ ID | +5G, +4C | ACGACUGAUGCCGUCGCG | 58.33 | 0 | 12.31 | 0 |
| NO. 57 | | | | | | |
| SEQ ID | +5U, −8C, −10G | GCCACUGAUGCCGUAUCG | 58.28 | 0 | 7.38 | 1.57 |
| NO. 58 | | | | | | |
| SEQ ID | +6G, +4G | ACGACUGAUGCCGUGAGG | 57.89 | 0.89 | 6.03 | 1.23 |
| NO. 59 | | | | | | |
| SEQ ID | +7C | ACGACUGAUGCCGUAACC | 57.89 | 0 | 9.66 | 0.37 |
| NO. 60 | | | | | | |
| SEQ ID | +7U, +2U, −5C | ACGACCGAUGCCUUAACU | 57.69 | 0 | 4.33 | 2.33 |
| NO. 61 | | | | | | |
| SEQ ID | +7C, +3G | ACGACUGAUGCCGGAACC | 57.69 | 0 | 15.56 | 0 |
| NO. 62 | | | | | | |
| SEQ ID | +7A, +3A, −8U | ACUACUGAUGCCGAAACA | 57.14 | 0 | 5.71 | 0 |
| NO. 63 | | | | | | |
| SEQ ID | +6G, +3A, −10G | GCGACUGAUGCCGAAAGG | 57.14 | 0 | 4.22 | 2.22 |
| NO. 64 | | | | | | |
| SEQ ID | +7U, +2C, −8A, | UAAACUGAUGCCCUAACU | 57.14 | 0 | 7.69 | 0 |
| NO. 65 | −9A, −10U | | | | | |
| SEQ ID | +7A, +6U, −4A | ACGACUAAUGCCGUAAUA | 57.14 | 0 | 8.22 | 1.28 |
| NO. 66 | | | | | | |
| SEQ ID | −9A, −10G | GAGACUGAUGCCGUAACG | 57.14 | 0 | 14.29 | 0 |
| NO. 67 | | | | | | |
| SEQ ID | +7A, +6A, +2C | ACGACUGAUGCCCUAAAA | 57.14 | 0 | 21.43 | 0 |
| NO. 68 | | | | | | |
| SEQ ID | +5C, +2C, −8U, | GCUACUGAUGCCCUACCG | 56.52 | 0 | 6.78 | 0 |
| NO. 69 | −10G | | | | | |
| SEQ ID | +5G, −8U | ACUACUGAUGCCGUAGCG | 56.52 | 0 | 6.99 | 0 |
| NO. 70 | | | | | | |
| SEQ ID | +4G, +2C, −5A | ACGACAGAUGCCCUGACG | 56.52 | 0 | 8.7 | 0 |
| NO. 71 | | | | | | |
| SEQ ID | +7U, +6G, +5G | ACGACUGAUGCCGUAGGU | 56.52 | 0 | 21.43 | 0 |
| NO. 72 | | | | | | |
| SEQ ID | +7U, +3C, −8A | ACAACUGAUGCCGCAACU | 56.48 | 0 | 8.7 | 32.61 |
| NO. 73 | | | | | | |
| SEQ ID | +7A, +5U, +2U, | ACGACCGAUGCCUUAUCA | 56.25 | 0 | 6.12 | 0 |
| NO. 74 | −5C | | | | | |
| SEQ ID | +7U | ACGACUGAUGCCGUAACU | 56.25 | 0.18 | 6.22 | 0.53 |
| NO. 75 | | | | | | |
| SEQ ID | +6U, +4C, −6G | ACGAGUGAUGCCGUCAUG | 56.1 | 1.89 | 3.77 | 0 |
| NO. 76 | | | | | | |
| SEQ ID | +7A, +3C | ACGACUGAUGCCGCAACA | 56 | 0 | 4.88 | 29.27 |
| NO. 77 | | | | | | |
| SEQ ID | +3A, +1U | ACGACUGAUGCUGAAACG | 55.81 | 0 | 6.82 | 6.52 |
| NO. 78 | | | | | | |
| SEQ ID | +7A, +6G, −8C | ACCACUGAUGCCGUAAGA | 55.56 | 0 | 5.56 | 0 |
| NO. 79 | | | | | | |
| SEQ ID | +5G, +2C | ACGACUGAUGCCCUAGCG | 55.56 | 0.21 | 4.97 | 1.69 |
| NO. 80 | | | | | | |
| SEQ ID | −8C, −10U | UCCACUGAUGCCGUAACG | 55.56 | 0 | 14.81 | 7.41 |
| NO. 81 | | | | | | |
| SEQ ID | +3C, −5C, −10U | UCGACCGAUGCCGCAACG | 55.56 | 0 | 3.7 | 37.04 |
| NO. 82 | | | | | | |
| SEQ ID | +6A, +3C | ACGACUGAUGCCGCAAAG | 55.56 | 0 | 6.67 | 37.78 |
| NO. 83 | | | | | | |
| SEQ ID | +6U, +3G, −4A | ACGACUAAUGCCGGAAUG | 55.26 | 0 | 5.56 | 0 |
| NO. 84 | | | | | | |
| SEQ ID | +3A, −8C | ACCACUGAUGCCGAAACG | 55.26 | 0 | 7.89 | 2.63 |
| NO. 85 | | | | | | |
| SEQ ID | +6A, +4U, +3G, | ACGACCGAUGCCGGUAAG | 55 | 0 | 9.21 | 1.23 |
| NO. 86 | −5C | | | | | |
| SEQ ID | +3C, −4A, −10G | GCGACUAAUGCCGCAACG | 55 | 0 | 7.89 | 28.95 |
| NO. 87 | | | | | | |
| SEQ ID | +7U, −5C | ACGACCGAUGCCGUAACU | 54.79 | 0 | 4.78 | 1.23 |
| NO. 88 | | | | | | |
| SEQ ID | +4C, +3A | ACGACUGAUGCCGACACG | 54.76 | 0 | 3.74 | 1.91 |
| NO. 89 | | | | | | |
| SEQ ID | +5° C., −10G | GCGACUGAUGCCGUACCG | 54.55 | 0 | 8.38 | 0 |
| NO. 90 | | | | | | |
| SEQ ID | +6A, −7G, −10G | GCGGCUGAUGCCGUAAAG | 54.55 | 0 | 9.09 | 0 |
| NO. 91 | | | | | | |
| SEQ ID | +4C, +3A, −6U | ACGAUUGAUGCCGACACG | 54.55 | 0 | 9.09 | 0 |
| NO. 92 | | | | | | |
| SEQ ID | +6A, +5G | ACGACUGAUGCCGUAGAG | 54.55 | 0 | 18.18 | 3.03 |
| NO. 93 | | | | | | |
| SEQ ID | +5G, +2C, −8U, | AAUACUGAUGCCCUAGCG | 54.39 | 0 | 9.09 | 0 |
| NO. 94 | −9A | | | | | |
| SEQ ID | +7U, −9U, −10U | UUGACUGAUGCCGUAACU | 54.17 | 0 | 28.57 | 0 |
| NO. 95 | | | | | | |
| SEQ ID | +4U, +1U, −6U, | GCAAUUGAUGCUGUUACG | 54.13 | 0 | 5.36 | 0 |
| NO. 96 | −8A, −10G | | | | | |
| SEQ ID | −5C, −8U | ACUACCGAUGCCGUAACG | 53.85 | 0 | 3.23 | 3.4 |
| NO. 97 | | | | | | |
| SEQ ID | +7A, −6A, −10U | UCGAAUGAUGCCGUAACA | 53.85 | 0 | 4.2 | 2.45 |
| NO. 98 | | | | | | |
| SEQ ID | +5C, +3A | ACGACUGAUGCCGAACCG | 53.85 | 0 | 12.45 | 0 |
| NO. 99 | | | | | | |
| SEQ ID | +4C, +3A, −8A | ACAACUGAUGCCGACACG | 53.85 | 0 | 15.38 | 0 |
| NO. 100 | | | | | | |
| SEQ ID | +5G, −4A | ACGACUAAUGCCGUAGCG | 53.85 | 3.1 | 12.41 | 2.75 |
| NO. 101 | | | | | | |
| SEQ ID | +3C, −6G, −10C | CCGAGUGAUGCCGCAACG | 53.85 | 0 | 0 | 30.77 |
| NO. 102 | | | | | | |

Table 2 shows examples of guide RNA sequences for editing LRRK2 G2019 with optimized antisense domains. Antisense sequences of guide RNAs from Table 1 that show the lowest off-target editing levels are ranked below based on their on-target editing levels.

TABLE 2

| | | | | | | | | | | | | | | | | | | | |
|---|---|---|---|---|---|---|---|---|---|---|---|---|---|---|---|---|---|---|---|
| SEQ ID NO: 1: | | | | | | A | C | U | A | C | A | G | C | A | U | U | G | C | |
| SEQ ID NO: 2: | U | G | C | U | G | A | C | U | A | C | A | G | C | A | U | U | G | C | Editing |
| Target Sequence | | | | | | | | | | | | | | | | | | | Level |
| (5′ to 3′) | | | | | | | | | | | | | | | | | | | (%) |
| Position | −10 | −9 | −8 | −7 | −6 | −5 | −4 | −3 | −2 | −1 | 0 | +1 | +2 | +3 | +4 | +5 | +6 | +7 | |
| relative to | | | | | | | | | | | | | | | | | | | |
| target Adenosine | | | | | | | | | | | | | | | | | | | |
| Prototype | A | C | G | A | C | U | G | A | U | G | C | C | G | U | A | A | C | G | 42.20 |
| antisense | | | | | | | | | | | | | | | | | | | |
| sequence | | | | | | | | | | | | | | | | | | | |
| (3′ to 5′) | | | | | | | | | | | | | | | | | | | |
| SEQ ID NO. 8: | G | C | G | A | C | U | G | A | U | G | C | C | G | U | A | A | C | C | 73.66 |
| +7C, -10G | | | | | | | | | | | | | | | | | | | |
| SEQ ID NO. 21: | A | C | A | A | C | C | G | A | U | G | C | C | G | A | A | A | C | G | 68.41 |
| +3A, -5C, -8A | | | | | | | | | | | | | | | | | | | |
| SEQ ID NO. 34: | A | C | G | A | C | U | C | A | U | G | C | C | G | U | A | A | G | G | 63.47 |
| +6G, -5C | | | | | | | | | | | | | | | | | | | |
| SEQ ID NO. 40: | A | C | G | A | C | C | C | A | U | G | C | C | G | U | A | A | C | C | 61.42 |
| +7C, -5C | | | | | | | | | | | | | | | | | | | |
| SEQ ID NO. 42: | G | C | G | A | C | U | G | A | U | G | C | C | G | U | C | A | G | G | 61.21 |
| +6G, +4C, -10G | | | | | | | | | | | | | | | | | | | |
| SEQ ID NO. 43: | A | C | G | A | C | U | G | A | U | G | C | C | A | U | A | A | C | C | 60.91 |
| +7C, +2A | | | | | | | | | | | | | | | | | | | |

Table 3 shows a list of recruitment sequences that may be incorporated into guide RNAs described herein. Recruitment sequences are designed to improve binding to target. Sequences arc numbered 3′ to 5′.

TABLE 3

| | Recruitment Sequence | Recruitment Sequence | Recruitment sequence |
|---|---|---|---|
| gRNA# | (RS) #3 | (RS) #2 | (RS) #1 |
| gRNA1 | SEQ ID NO: 105 | SEQ ID NO: 106 | SEQ ID NO: 107 |
| | AAGCAGGCGAUCCAA | UAUCUCAAACCAUCA | UGAAAAGCAGCACAU |
| | GGAAC | | |
| gRNA2 | SEQ ID NO: 108 | SEQ ID NO: 109 | SEQ ID NO: 110 |
| | GUGUGAAAAGCAGCA | GGUAUCUCAAACCAU | GUGUGAAAAGCAGCA |
| | CAUUG | CAGCU | CAUUG |
| gRNA3 | SEQ ID NO: 111 | SEQ ID NO: 112 | SEQ ID NO: 113 |
| | GUCAGCCAAAAUCAA | AACUCCAUCACCAAC | AGCAGGCGAUCCAAG |
| | GUCAG | AUCCG | GAACC |
| gRNA4 | SEQ ID NO: 114 | SEQ ID NO: 115 | SEQ ID NO: 116 |
| | CAUCCGGGGACGAAU | CGAUCCAAGGAACCC | UCUCAAACCAUCAGC |

Exemplary sequences for the antisense domain that can be used in the fusion constructs described herein are as follows:

Sequence 1 (Placeholder sequence provides support for all substitutions) from 3′ to 5′
X1-X2-X3-X4-X5-X6-X7-X8-X9-X10-C-X11-X12-X13-X14-X15-X16-X17
Sequence 2 (Generic −5C feature)
X1-X2-X3-X4-X5-C-X7-X8-X9-X10-C-X11-X12-X13-X14-X15-X16-X17
Sequence 3: (Generic +3C feature)
X1-X2-X3-X4-X5-X6-X7-X8-X9-X10-C-X11-X12-C-X14-X15-X16-X17
Sequence 4: (Generic +7C feature)
X1-X2-X3-X4-X5-X6-X7-X8-X9-X10-C-X11-X12-X13-X14-X15-X16-C
Sequence 5: (+7C, +2A Generic Sequence)
X1-X2-X3-X4-X5-X6-X7-X8-X9-X10-C-X11-A-X13-X14-X15-X16-C
Sequence 6: (+7C, +2A Sequence)
ACGACUGAUGCCAUAACC
Sequence 7: (+6G, +4C, −10G Generic Sequence)
G-X2-X3-X4-X5-X6-X7-X8-X9-X10-C-X11-X12-X13-C-X15-C-X17
Sequence 8: (+6G, +4C, −10G Sequence)
GCGACUGAUGCCGUCAGG
Sequence 9: (+7C, −5C Generic Sequence)
X1-X2-X3-X4-X5-C-X7-X8-X9-X10-C-X11-X12-X13-X14-X15-X16-C
Sequence 10: (+7C, −5C Sequence)
ACGACCGAUGCCGUAACC
Sequence 11: (+6G, −5C Generic Sequence)
X1-X2-X3-X4-X5-C-X7-X8-X9-X10-C-X11-X12-X13-X14-X15-G-X17
Sequence 12: (+6G, −5C Sequence)
ACGACCGAUGCCGUAAGG
Sequence 13: (+7C, −10G Generic Sequence)
G-X2-X3-X4-X5-X6-X7-X8-X9-X10-C-X11-X12-X13-X14-X15-X16-C
Sequence 14: (+7C, −10G Sequence)
GCGACUGAUGCCGUAACC

Mismatches that De-Enrich Editing (Negative Rules) were Discovered to be:
−7G, −2G, −1C, −1T, 0A, 0G, +1A

This is shown by the heatmap provided as FIG. 6.

### REFERENCES

Akhtar S, and Juliano R L, (1992) Trends Cell. Biol. 2 (5): 139-144.
Alessi D R and Sammler E (2018). LRRK2 kinase in Parkinson's disease. Science 360:36-37.
Baptista MAS, Dave K D, Frasier M A, Sherer T B, Greeley M, Beck M J, Varsho J S, Parker G A, Moore C, Churchill M J, et al (2013). Loss of leucine-rich repeat kinase 2 (LRRK2) in rats leads to progressive abnormal phenotypes in peripheral organs. PLOS One 8: e80705.
Bardien S, Lesage S, Brice A, Carr J. (2011). Genetic characteristics of leucine-rich repeat kinase 2 (LRRK2) associated Parkinson's disease. Parkinsonism Relat Disord. 17:501-508.
Bass, B L and Weintraub H. (1987). A developmentally regulated activity that unwinds RNA duplexes. Cell. 48:607-13.
Bieri G, Brahic M, Bousset L, Couthouis J, Kramer N J, Ma R, Nakayama L, Monbureau M, Defensor E, Schüle B, et al (2019). LRRK2 modifies a-syn pathology and spread in mouse models and human neurons. Acta Neuropathol 137:961-980.
Buenrostro J D, Araya C L, Chircus L M, Layton C J, Chang H Y, Snyder M P & Greenleaf W J (2014). Quantitative analysis of RNA-protein interactions on a massively parallel array reveals biophysical and evolutionary landscapes. Nature Biotechnology 32:562-568.
Coppedè F (2012). Genetics and Epigenetics of Parkinson's Disease. The Scientific World Journal. Volume 2012, Article I D 489830.
Debacker, A. J.; Voutila, J.; Catley, M.; Blkey, D.; and Habib, N. (2020). Delivery of Oligonucleotides to the Liver with GalNAc: From Research to Registered Therapeutic Drug. Molecular Therapy. 28 (8): 1759-1771.
Fuji R N, Flagella M, Baca M, Baptista MAS, Brodbeck J, Chan B K, Fiske B K, Honigberg L, Jubb A M, Katavolos P, et al (2015). Effect of selective LRRK2 kinase inhibition on nonhuman primate lung. Sci Transl Med 7: 273ra15
Jennings D, Huntwork-Rodriguez S, Henry A G, Sasaki J C, Meisner R, Diaz D, Solanoy H, Wang X, Negrou E, Bondar V V, et al (2022). Preclinical and clinical evaluation of the LRRK2 inhibitor DNL201 for Parkinson's disease. Sci Transl Med 14: eabj2658.
Kim, U., Wang, Y., Sanford, T., Zeng, Y., Nishikura, K. (1994). Molecular cloning of cDNA for double-stranded RNA adenosine deaminase, a candidate enzyme for nuclear RNA editing. Proc. Nat. Acad. Sci. 91:11457-11461.
Kivioja T, Vähärautio A, Karlsson K, Bonke M, Enge M, Linnarsson S & Taipale J (2012). Counting absolute numbers of molecules using unique molecular identifiers. Nature Methods 9:72-74.
Kumari U, and Tan E K (2009). LRRK2 in Parkinson's disease: genetic and clinical studies from patients. FEBS J. 276 (22): 6455-6463.
Levin A A (2019). Treating Disease at the RNA Level with Oligonucleotides. New England Journal of Medicine 380:57-70.
Li Jie-Qiong, Tan L., & Jin-Tai Yu (2014). The role of the LRRK2 gene in Parkinsonism. Molecular Neurodegeneration. 9 (47): https://doi.org/10.1186/1750-1326-9-47.
Lis P, Burel S, Steger M, Mann M, Brown F, Diez F, Tonelli F, Holton J L, Ho P W, Ho S-L, et al (2018). Development of phospho-specific Rab protein antibodies to monitor in vivo activity of the LRRK2 Parkinson's disease kinase. Biochem J, 475:1-22.
Merkle T, Merz S, Reautschnig P, Blaha A, Li Q, Vogel P, Wettengel J, Li J B & Stafforst T (2019). Precise RNA editing by recruiting endogenous ADARs with antisense oligonucleotides. Nature Biotechnology, 37:133-138.
Mhyre T R, Boyd J T, Hamill R W, & Maguire-Zeiss, K A. (2012). Parkinson's Disease. Subcell Biochem. 65:389-455.
Monian, P.; Shivalila, C.; Lu, G.; Shimizu; M.; Boulay, D.; Bussow, K.; Byrne, M.; Bezigian, A.; Chatterjee, A.; Chew, D.; Desai, J.; Favaloro, F.; Godfrey, F.; Hoss, A.; et al. (2022). Endogenous ADAR-mediated RNA editing in non-human primates using stereopure chemically modified oligonucleotides. Nature Biotech. 40:1093-1102.
Nishikura K (2016). A-to-I editing of coding and non-coding RNAs by ADARs. Nature Reviews Molecular Cell Biology, 17:83-96.
Ramaswami G, Zhang R, Piskol R, Keegan L P, Deng P, O'Connell M A & Li J B (2013). Identifying RNA editing sites using RNA sequencing data alone. Nat Methods 10:128-132.
Reautschnig P, Wahn N, Wettengel J, Schulz A E, Latifi N, Vogel P, Kang T-W, Pfeiffer L S, Zarges C, Naumann U, et al (2022). CLUSTER guide RNAs enable precise and efficient RNA editing with endogenous ADAR enzymes in vivo. Nat Biotechnol: 1-10
Rebagliati, M. R.; Melton D. A. (1987). Antisense RNA injections in fertilized frog eggs reveal an RNA duplex unwinding activity. Cell. 48:599-605.
Steger M, Diez F, Dhekne H S, Lis P, Nirujogi R S, Karayel O, Tonelli F, Martinez T N, Lorentzen E, Pfeffer S R, et al (2017). Systematic proteomic analysis of LRRK2-mediated Rab GTPase phosphorylation establishes a connection to ciliogenesis. eLife 6: e31012.
Steger M, Tonelli F, Ito G, Davies P, Trost M, Vetter M, Wachter S, Lorentzen E, Duddy G, Wilson S, et al (2016). Phosphoproteomics reveals that Parkinson's disease kinase LRRK2 regulates a subset of Rab GTPases. eLife 5.
Tan M H, Li Q, Shanmugam R, Piskol R, Kohler J, Young A N, Liu K I, Zhang R, Ramaswami G, Ariyoshi K, et al (2017). Dynamic landscape and regulation of RNA editing in mammals. Nature 550:249-254.
Thomas, J. M., and Beal, P. A. (2017). How do ADARs bind RNA? New protein-RNA structures illuminate substrate recognition by the RNA editing ADARs. Bioassays. 39 (4): 1-17.
Tomaselli, S.; Locatelli, F.; Gallo, A. (2014). The RNA editing enzymes ADARs: mechanism of action and human disease. Cell and Tissue Research. 356:527-532.
Wong, S K, Sato S, and Lazinski, D W. (2001). Substrate recognition by ADAR1 and ADAR2. RNA. 7:846-858.
Wulff, B.-E., and Nishikura, K. (2010). Substitutional A-to-I RNA editing. Interdiscip. Rev. RNA. 1 (1): 90-101.
Zinshteyn, B, Nishikura, K. (2009). Adenosine-to-inosine RNA editing. WIREs Systems Biology and Medicine. 1 (2): 202-209.

1. An antisense oligonucleotide suitable for editing an adenosine in a target RNA nucleic acid sequence to inosine (A-to-I editing), wherein
the antisense oligonucleotide comprises a sequence that is substantially complementary to the target RNA nucleic acid sequence:
(SEQ ID NO: 1)
5′-ACUACAGCAUUGC 3′,
or complementary to a sequence that has at least 80% sequence identity to SEQ ID NO: 1, wherein A is the target adenosine to be edited in the target nucleic acid; and the antisense oligonucleotide can be represented by: 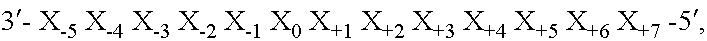
wherein X represents a nucleic acid, X₀ represents cytosine (C), deoxycytosine (dC), or uracil (U) directly opposite to the target adenosine to be edited, and wherein there is a mismatch between the antisense oligonucleotide and the target nucleic acid at one or more of the following position(s):
a)−5 (X₋₅);
b)+3 (X₊₃);
c)+7 (X₊₇);
d)+7 (X₊₇) and +2 (X₊₂);
e)+7 (X₊₇) and −5 (X₋₅); or
f)+6 (X₊₆) and −5 (X₋₅).

2. An antisense oligonucleotide suitable for editing an adenosine in a target RNA nucleic acid sequence to inosine (A-to-I editing), wherein
the antisense oligonucleotide comprises a sequence that is substantially complementary to the target RNA nucleic acid sequence:
(SEQ ID NO: 2)
5′-UGCUGACUACAGCAUUGC-3′,
or complementary to a sequence that has at least 80% sequence identity to SEQ ID NO: 2, wherein A is the target adenosine to be edited in the target nucleic acid; and the antisense oligonucleotide can be represented by: 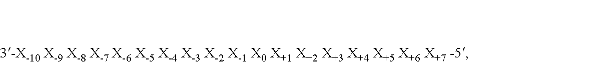
wherein X represents a nucleic acid, X₀ represents cytosine (C), deoxycytosine (dC), or uracil (U) directly opposite to the target adenosine to be edited, and wherein there is a mismatch between the antisense oligonucleotide and the target nucleic acid at one or more of the following position(s):
g)+6 (X₊₆) and −5 (X₋₅);
h)+7 (X₊₇) and −10 (X₋₁₀); or
i)+3 (X₊₃), −5 (X₋₅), and −8 (X₋₈).

3. The antisense oligonucleotide of claim 1, wherein the oligonucleotide comprises a sequence that is substantially complementary to the target RNA nucleic acid sequence of SEQ ID NO:
2, optionally wherein there is a mismatch between the antisense oligonucleotide and the target nucleic acid at one or more of the following position(s):
g)+6 (X₊₆) and −5 (X₋₅);
h)+7 (X₊₇) and −10 (X₋₁₀); or
i)+3 (X₊₃), −5 (X₋₅), and −8 (X₋₈).

4. The antisense oligonucleotide of any one of claims 1-3, wherein the target RNA nucleic acid sequence comprises a nucleotide sequence having at least 85%, at least 90%, at least 91%, at least 92%, at least 93%, at least 94%, at least 95%, at least 96%, at least 97%, at least 98%, at least 99%, or 100% identity to SEQ ID NO: 1 or 2.

5. The antisense oligonucleotide of any one of claims 1-4, wherein the mismatch is a paired C-A, C-C, A-C, G-G, C-U or G-U mismatch.

6. The antisense oligonucleotide of claim 1, wherein the one or more nucleotides that are not complementary to the nucleotide(s) in the corresponding location in the target nucleic acid is:
a) cytosine (−5C) at position −5 (X₋₅);
b) cytosine (+3C) at position +3 (X₊₃);
c) cytosine (+7C) at position +7 (X₊₇);
d) cytosine at position +7 (X₊₇) and adenosine at position +2 (X₊₂) is (+7C and +2A);
e) cytosine at position +7 (X₊₇) and cytosine at position −5 (X₋₅)(+7C and −5C); or
f) guanosine at position +6 (X₊₆) and cytosine at position −5 (X₋₅)(+6G and −5C).

7. The antisense oligonucleotide of any one of claims 2-5, wherein the one or more nucleotides that are not complementary to the nucleotide(s) in the corresponding location in the target nucleic acid is:
a) cytosine (−5C) at position −5 (X₋₅);
b) cytosine (+3C) at position +3 (X₊₃);
c) cytosine (+7C) at position +7 (X₊₇);
d) cytosine at position +7 (X₊₇) and adenosine at position +2 (X₊₂) is (+7C and +2A);
e) cytosine at position +7 (X₊₇) and cytosine at position −5 (X₋₅)(+7C and −5C);
f) guanosine at position +6 (X₊₆) and cytosine at position −5 (X₋₅)(+6G and −5C);
g) cytosine at position +7 (X₊₇) and guanosine at position −10 (X₋₁₀)(+7C and −10G);
h) adenosine at position +3 (X₊₃), cytosine at position −5 (X₋₅), and adenosine at position −8 (X₋₈)(+3A,-5C, and −8A); or
i) guanosine at position +6 (X₊₆), cytosine at position +4 (X₊₄), and guanosine at position −10 (X₋₁₀)(+6G, +4C and −10G).

8. The antisense oligonucleotide of claim 2-7, wherein the antisense oligonucleotide comprises the general core sequence selected from the list of: 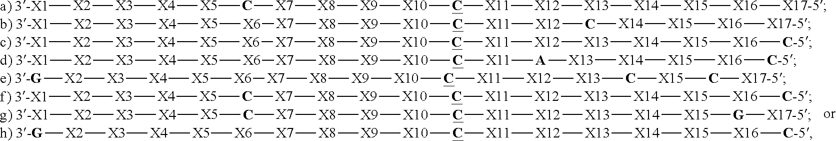
wherein G, C, and A correspond to guanosine, cytosine and adenosine respectively; and
wherein C corresponds to the central nucleotide (X₀) directly opposite the target adenosine (A) to be edited when the antisense domain is hybridised to the target nucleic acid.

9. The antisense oligonucleotide of claim 1-8, wherein the antisense oligonucleotide comprises at least 80%, at least 85% or at least 90% sequence complementarity to the target nucleic acid sequence.

10. The antisense oligonucleotide of claim 2-9, wherein the antisense oligonucleotide comprises a sequence selected from the group consisting of:
a) SEQ NO: 8,
b) SEQ NO: 21,
c) SEQ NO: 34,
d) SEQ NO: 40,
e) SEQ NO: 42, and
f) SEQ NO: 43.

11. The antisense oligonucleotide of any one of claims 1-10, wherein the oligonucleotide does not comprise one or more of the following mismatches selected from the group consisting of: −7G, −2G, −1C, −1T, 0A, 0G, and +1A.

12. The antisense oligonucleotide of any one of claims 1-11, wherein the nucleic acid sequence of the oligonucleotide comprises at least 20, 25, 30, 35, or 45 nucleotides (nt).

13. The antisense oligonucleotide of any one of claims 1-12, wherein the nucleic acid sequence of the oligonucleotide is 15-80 nt, preferably 24-45 nt, most preferably 30-35 nt in length.

14. The antisense oligonucleotide of any one of claims 1-13, wherein the oligonucleotide comprises at least 1 recruitment sequence (RS).

15. The antisense oligonucleotide of any one of claims 1-14, wherein the oligonucleotide comprises 1, 2 or 3 recruitment sequences.

16. The antisense oligonucleotide of claim 15, wherein:
(I) a) a first recruitment sequence is 3′-UGAAAAGCAGCACAU-5′ (SEQ ID NO: 107),
b) a second recruitment sequence is 3′-UAUCUCAAACCAUCA-5′ (SEQ ID NO: 106), and
c) a third recruitment sequence is 3′-AAGCAGGCGAUCCAAGGAAC-5′ (SEQ ID NO: 105);
(II) a) a first recruitment sequence is 3′-GUGUGAAAAGCAGCACAUUG-5′ (SEQ ID NO: 110),
b) a second recruitment sequence is 3′-GGUAUCUCAAACCAUCAGCU-5′ (SEQ ID NO: 109), and
c) a third recruitment sequence is 3′-GUGUGAAAAGCAGCACAUUG-5′ (SEQ ID NO: 108);
(III) a) a first recruitment sequence is 3′-AGCAGGCGAUCCAAGGAACC-5′ (SEQ ID NO: 113),
b) a second recruitment sequence is 3′-AACUCCAUCACCAACAUCCG-5′ (SEQ ID NO: 112), and
c) a third recruitment sequence is 3′-GUCAGCCAAAAUCAAGUCAG-5′ (SEQ ID NO: 111); or
(IV) a) a first recruitment sequence is 3′-UCUCAAACCAUCAGC-5′ (SEQ ID NO: 116),
b) a second recruitment sequence is 3′-CGAUCCAAGGAACCC-5′ (SEQ ID NO: 115), and
c) a third recruitment sequence is 3′-CAUCCGGGGACGAAU-5′ (SEQ ID NO: 114).

17. The antisense oligonucleotide of any one of claims 1-16, wherein the antisense oligonucleotide does not comprise a target sequence.

18. The antisense oligonucleotide of any one of claims 1-17, wherein the target RNA nucleic acid encodes a LRRK2 polypeptide comprising a mutation corresponding to a G2019S mutation.

19. The antisense oligonucleotide of any one of claims 1-18, wherein the antisense oligonucleotide comprises at least one chemical modification.

20. The antisense oligonucleotide of claim 19, wherein 20-100% of nucleotides are 2′-modified, preferably wherein 50-100% of nucleotides are 2′-modified nucleotides.

21. The antisense oligonucleotide of claim 19 or 20, wherein
a) 20-70% of nucleotides are 2′-Fluoro (F)-modified, preferably wherein 35-65% of nucleotides are 2′-F-modified; and/or
b) 20-60% of nucleotides are 2′-O-methyl(2′-OMe)-modified, preferably wherein 25-55% of nucleotides are 2′-OMe-modified.

22. The antisense oligonucleotide of any one of claims 19-21, wherein
(i) no more than 95%, 90%, 85%, 80%, 70%, 60%, 50%, 40%, 30% or 20% of the linkages are internucleoside linkage modifications; or
(ii) 15-90% of the linkages are internucleoside linkage modifications, preferably wherein 40-80%, most preferably 45-60%, of the linkages are internucleoside linkage modifications.

23. The antisense oligonucleotide of any one of claims 19-22, comprising at least one internucleoside linkage modification selected from the group consisting of phosphorothioate (PS), 3′-methylenephosphonate, 5′-methylenephosphonate, 3′-phosphoroamidate, 2′-5′phosphodiester, and phosphoryl guanidine (PN), preferably wherein the at least one internucleoside linkage modification is PS.

24. The antisense oligonucleotide of claim 23, wherein the antisense oligonucleotide comprises:
a) at least 60% of PS linkages;
b) 60%-95% PS linkages.

25. The antisense oligonucleotide any one of claims 19-24, wherein
(i) no more than 4, 5, or 6 consecutive nucleotides are 2′-F-modified; and/or
(ii) no more than 4, 5, or 6 consecutive nucleotides are 2′-OMe-modified.

26. The antisense oligonucleotide according to any one of claims 19-25, wherein less than 6, 5, 4, or 3 consecutive nucleotides have the same 2′-modification.

27. The antisense oligonucleotide any one of claims 1-26, wherein
(i) X₊₁ is 2′-F, 2′-FANA, DNA, or 2′-O-methyl; and/or
(ii) X₀ is 2′-FANA or DNA; and/or
(iii) X₋₁ is 2′-FANA, DNA, or 2′-O-methyl.

28. The antisense oligonucleotide any one of claims 19-27, wherein the X₋₂ nucleotide carries a 2′-O-alkyl-modification, optionally wherein the 2′-O-alkyl-modification is a 2′-O-methyl (2′-OMe)-modification; and/or wherein the X-3 nucleotide carries a 2′-fluoro (2′-F)-modification.

29. The antisense oligonucleotide any one of claims 1-28, wherein the oligonucleotide does not comprise a hairpin-loop structured ADAR recruitment motif or domain.

30. A vector comprising the antisense oligonucleotide of any one of claims 1-29.

31. The vector of claim 30, wherein the vector is a viral vector or non-viral vector.

32. The vector of claim 31, wherein the viral vector is a lentiviral vector, retroviral vector, adenovirus vector, adeno-associated virus vector, or alphavirus vector.

33. The vector of claim 31, wherein the non-viral vector is a plasmid, cosmid, or phage.

34. A pharmaceutical composition or a pharmaceutically acceptable salt thereof comprising an antisense oligonucleotide of any one of claims 1-29, or a vector of any one of claims 30-33.

35. A method for site-directed A-to-I editing of a target RNA, comprising providing to a cell or subject an oligonucleotide according to any one of claims 1-29, a vector of any one of claims 30-33 or a pharmaceutical composition according to claim 34.

36. An in vitro method for deaminating at least one specific adenosine present in a target RNA sequence in a cell, wherein the method comprises the steps of:
(a) contacting the target nucleic acid with an antisense oligonucleotide of any one of claims 1-29, a vector of any one of claims 30-33 or a pharmaceutical composition according to claim 34;
(b) allowing uptake by the cell of the antisense oligonucleotide;
(c) allowing annealing of the antisense domain to the target RNA sequence; and
(d) allowing a mammalian ADAR enzyme comprising a natural dsRNA binding domain as found in the wild-type enzyme to deaminate the target adenosine in the target RNA sequence to an inosine.

37. The method of claim 36, wherein the cell endogenously expresses ADAR.

38. The method of claim 36 or 37, wherein the ADAR is a human ADAR.

39. The method of any one of claims 36-38, wherein the ADAR is human ADAR1.

40. A method of treating a subject suffering from a genetic disorder, condition, or disease, wherein the method comprises administering to the subject in need thereof an effective amount of an antisense oligonucleotide of any one of claims 1-29, a vector of any one of claims 30-33 or pharmaceutically acceptable salt thereof, or a pharmaceutical composition of claim 34.

41. The method of claim 40, wherein the genetic disorder, condition, or disease is a LRRK2-associated disease or condition.

42. The method of claim 41, wherein the LRRK2-associated disease or condition is associated with a LRRK2 G2019S mutation.

43. The method of any one of claims 40-42, wherein the genetic disorder, condition, or disease is Parkinson's Disease (P D), Lewy body dementia, frontotemporal dementia, corticobasal 3 dementia, progressive supranuclear palsy, Alzheimer's disease, tauopathy disease, or alpha-4 synucleinopathy.

44. The method of any one of claims 40-43, wherein the subject is a human subject.

45. An antisense oligonucleotide of any one of claims 1-29, a vector of any one of claims 30-33 or a pharmaceutical composition of claim 34 for use in therapy.

46. An antisense oligonucleotide of any one of claims 1-29, a vector of any one of claims 30-33 or a pharmaceutical composition of claim 34 for use in the treatment of a disease or disorder, where in the disease or disorder is selected form the group consisting of Parkinson's Disease (PD), Lewy body dementia, frontotemporal dementia, corticobasal 3 dementia, progressive supranuclear palsy, Alzheimer's disease, tauopathy disease, or alpha-4 synucleinopathy.

47. The antisense oligonucleotide, the vector or the composition for use of claim 46, wherein the disease or disorder comprises the Irrk2 gene or transcript thereof.

48. A fusion construct comprising a target sequence and a guide RNA sequence, wherein the target sequence comprises a sequence from the LRRK2 gene having a G2019S mutation and wherein the guide RNA sequence comprises an antisense domain that is substantially complementary or perfectly complementary to the target sequence.

49. The fusion construct of claim 48, wherein:
a. the antisense sequence comprises a sequence shown in Table 1;
b. the antisense sequence comprises a sequence shown in Table 2;
c. the target sequence comprises SEQ ID NO: 1 or SEQ ID NO: 2 and the antisense sequence has at least one, two, three, four, five, six, or more mismatches when hybridized to target sequence; or
d. the antisense sequence comprises an amino acid sequence having at least 80% identity to SEQ ID NO: 6.

50. The fusion construct of claim 48 or claim 49, wherein the antisense sequence is joined to one or more recruitment sequences shown in Table 3.

51. A method of editing the LRRK2 G2019S mutation in a subject having Parkinson's Disease (PD), comprising expressing in or administering to a cell of the subject the fusion construct of claim 48, 49, or 50.


SEQUENCE LISTING (116 sequences)

SEQ ID NO: 1 - RNA, 13 nt, synthetic construct
  source 1..13: mol_type = other RNA
actacagcattgc

SEQ ID NO: 2 - RNA, 18 nt, synthetic construct
  source 1..18: mol_type = other RNA
tgctgactacagcattgc

SEQ ID NO: 3 - RNA, 18 nt, synthetic construct
  source 1..18: mol_type = other RNA
acgactggtgccgcaact

SEQ ID NO: 4 - RNA, 18 nt, synthetic construct
  source 1..18: mol_type = other RNA
ccgactaatgccgctact

SEQ ID NO: 5 - RNA, 18 nt, synthetic construct
  source 1..18: mol_type = other RNA
acgactgattccgaaacg

SEQ ID NO: 6 - RNA, 18 nt, synthetic construct
  source 1..18: mol_type = other RNA
atgactgatgccgcaatg

SEQ ID NO: 7 - RNA, 18 nt, synthetic construct
  source 1..18: mol_type = other RNA
ccgactgatgccgcaacg

SEQ ID NO: 8 - RNA, 18 nt, synthetic construct
  source 1..18: mol_type = other RNA
gcgactgatgccgtaacc

SEQ ID NO: 9 - RNA, 18 nt, synthetic construct
  source 1..18: mol_type = other RNA
acgaccgatgccggaagg

SEQ ID NO: 10 - RNA, 18 nt, synthetic construct
  source 1..18: mol_type = other RNA
aggagtgatgccgcaacg

SEQ ID NO: 11 - RNA, 18 nt, synthetic construct
  source 1..18: mol_type = other RNA
aagagtgatgccgcaaca

SEQ ID NO: 12 - RNA, 18 nt, synthetic construct
  source 1..18: mol_type = other RNA
ctgactgatgccttaacg

SEQ ID NO: 13 - RNA, 18 nt, synthetic construct
  source 1..18: mol_type = other RNA
acgactcatgccgtgatg

SEQ ID NO: 14 - RNA, 18 nt, synthetic construct
  source 1..18: mol_type = other RNA
aggactgatgccgtgccg

SEQ ID NO: 15 - RNA, 18 nt, synthetic construct
  source 1..18: mol_type = other RNA
gcgactgatgccgtaact

SEQ ID NO: 16 - RNA, 18 nt, synthetic construct
  source 1..18: mol_type = other RNA
acgactgatgccctaacc

SEQ ID NO: 17 - RNA, 18 nt, synthetic construct
  source 1..18: mol_type = other RNA
acgactgatgccctaggt

SEQ ID NO: 18 - RNA, 18 nt, synthetic construct
  source 1..18: mol_type = other RNA
acgactgatgccgcaatt

SEQ ID NO: 19 - RNA, 18 nt, synthetic construct
  source 1..18: mol_type = other RNA
gtgactgatgccgcaacc

SEQ ID NO: 20 - RNA, 18 nt, synthetic construct
  source 1..18: mol_type = other RNA
gcgactgatgccgcaaag

SEQ ID NO: 21 - RNA, 18 nt, synthetic construct
  source 1..18: mol_type = other RNA
acaaccgatgccgaaacg

SEQ ID NO: 22 - RNA, 18 nt, synthetic construct
  source 1..18: mol_type = other RNA
ccgactgatgccgtcacc

SEQ ID NO: 23 - RNA, 18 nt, synthetic construct
  source 1..18: mol_type = other RNA
aagactgatgccgtaacc

SEQ ID NO: 24 - RNA, 18 nt, synthetic construct
  source 1..18: mol_type = other RNA
acgactaatgccgtaccg

SEQ ID NO: 25 - RNA, 18 nt, synthetic construct
  source 1..18: mol_type = other RNA
acgcctgacgcctcaacc

SEQ ID NO: 26 - RNA, 18 nt, synthetic construct
  source 1..18: mol_type = other RNA
atgactgatgccgttccg

SEQ ID NO: 27 - RNA, 18 nt, synthetic construct
  source 1..18: mol_type = other RNA
acgactgatgccgttacc

SEQ ID NO: 28 - RNA, 18 nt, synthetic construct
  source 1..18: mol_type = other RNA
acgactgatgccggaaca

SEQ ID NO: 29 - RNA, 18 nt, synthetic construct
  source 1..18: mol_type = other RNA
acgactaatgccgtaact

SEQ ID NO: 30 - RNA, 18 nt, synthetic construct
  source 1..18: mol_type = other RNA
agcaccgatgccgtaacg

SEQ ID NO: 31 - RNA, 18 nt, synthetic construct
  source 1..18: mol_type = other RNA
acgactgatgccgttccg

SEQ ID NO: 32 - RNA, 18 nt, synthetic construct
  source 1..18: mol_type = other RNA
acgactgatgccgtatcc

SEQ ID NO: 33 - RNA, 18 nt, synthetic construct
  source 1..18: mol_type = other RNA
gccactgatgccgtagcg

SEQ ID NO: 34 - RNA, 18 nt, synthetic construct
  source 1..18: mol_type = other RNA
acgaccgatgccgtaagg

SEQ ID NO: 35 - RNA, 18 nt, synthetic construct
  source 1..18: mol_type = other RNA
actactgatgccgcaagg

SEQ ID NO: 36 - RNA, 18 nt, synthetic construct
  source 1..18: mol_type = other RNA
acgacagatgccgcaact

SEQ ID NO: 37 - RNA, 18 nt, synthetic construct
  source 1..18: mol_type = other RNA
tcgagtgatgccgtgaag

SEQ ID NO: 38 - RNA, 18 nt, synthetic construct
  source 1..18: mol_type = other RNA
acgaccgatgccgtaaca

SEQ ID NO: 39 - RNA, 18 nt, synthetic construct
  source 1..18: mol_type = other RNA
cagaccgatgccgtaacg

SEQ ID NO: 40 - RNA, 18 nt, synthetic construct
  source 1..18: mol_type = other RNA
acgaccgatgccgtaacc

SEQ ID NO: 41 - RNA, 18 nt, synthetic construct
  source 1..18: mol_type = other RNA
acgactgatgccttggcg

SEQ ID NO: 42 - RNA, 18 nt, synthetic construct
  source 1..18: mol_type = other RNA
gcgactgatgccgtcagg

SEQ ID NO: 43 - RNA, 18 nt, synthetic construct
  source 1..18: mol_type = other RNA
acgactgatgccataacc

SEQ ID NO: 44 - RNA, 18 nt, synthetic construct
  source 1..18: mol_type = other RNA
accactgatgccgtgaga

SEQ ID NO: 45 - RNA, 18 nt, synthetic construct
  source 1..18: mol_type = other RNA
acgaccgatgccgtagcg

SEQ ID NO: 46 - RNA, 18 nt, synthetic construct
  source 1..18: mol_type = other RNA
aggactgatgccgcaacc

SEQ ID NO: 47 - RNA, 18 nt, synthetic construct
  source 1..18: mol_type = other RNA
ccgaccgatgccgtaacc

SEQ ID NO: 48 - RNA, 18 nt, synthetic construct
  source 1..18: mol_type = other RNA
acgaccgatgctgcaacg

SEQ ID NO: 49 - RNA, 18 nt, synthetic construct
  source 1..18: mol_type = other RNA
acgactgttgccgtaccg

SEQ ID NO: 50 - RNA, 18 nt, synthetic construct
  source 1..18: mol_type = other RNA
acgactgatgccgcaact

SEQ ID NO: 51 - RNA, 18 nt, synthetic construct
  source 1..18: mol_type = other RNA
aggactgatgccgtaacc

SEQ ID NO: 52 - RNA, 18 nt, synthetic construct
  source 1..18: mol_type = other RNA
aggaccgatgccgtaacc

SEQ ID NO: 53 - RNA, 18 nt, synthetic construct
  source 1..18: mol_type = other RNA
acgactgatgccctaaca

SEQ ID NO: 54 - RNA, 18 nt, synthetic construct
  source 1..18: mol_type = other RNA
gcgactgatgccgtaaca

SEQ ID NO: 55 - RNA, 18 nt, synthetic construct
  source 1..18: mol_type = other RNA
tcgactgatgccgtaaca

SEQ ID NO: 56 - RNA, 18 nt, synthetic construct
  source 1..18: mol_type = other RNA
ccgactgatgccgtaacc

SEQ ID NO: 57 - RNA, 18 nt, synthetic construct
  source 1..18: mol_type = other RNA
acgactgatgccgtcgcg

SEQ ID NO: 58 - RNA, 18 nt, synthetic construct
  source 1..18: mol_type = other RNA
gccactgatgccgtatcg

SEQ ID NO: 59 - RNA, 18 nt, synthetic construct
  source 1..18: mol_type = other RNA
acgactgatgccgtgagg

SEQ ID NO: 60 - RNA, 18 nt, synthetic construct
  source 1..18: mol_type = other RNA
acgactgatgccgtaacc

SEQ ID NO: 61 - RNA, 18 nt, synthetic construct
  source 1..18: mol_type = other RNA
acgaccgatgccttaact

SEQ ID NO: 62 - RNA, 18 nt, synthetic construct
  source 1..18: mol_type = other RNA
acgactgatgccggaacc

SEQ ID NO: 63 - RNA, 18 nt, synthetic construct
  source 1..18: mol_type = other RNA
actactgatgccgaaaca

SEQ ID NO: 64 - RNA, 18 nt, synthetic construct
  source 1..18: mol_type = other RNA
gcgactgatgccgaaagg

SEQ ID NO: 65 - RNA, 18 nt, synthetic construct
  source 1..18: mol_type = other RNA
taaactgatgccctaact

SEQ ID NO: 66 - RNA, 18 nt, synthetic construct
  source 1..18: mol_type = other RNA
acgactaatgccgtaata

SEQ ID NO: 67 - RNA, 18 nt, synthetic construct
  source 1..18: mol_type = other RNA
gagactgatgccgtaacg

SEQ ID NO: 68 - RNA, 18 nt, synthetic construct
  source 1..18: mol_type = other RNA
acgactgatgccctaaaa

SEQ ID NO: 69 - RNA, 18 nt, synthetic construct
  source 1..18: mol_type = other RNA
gctactgatgccctaccg

SEQ ID NO: 70 - RNA, 18 nt, synthetic construct
  source 1..18: mol_type = other RNA
actactgatgccgtagcg

SEQ ID NO: 71 - RNA, 18 nt, synthetic construct
  source 1..18: mol_type = other RNA
acgacagatgccctgacg

SEQ ID NO: 72 - RNA, 18 nt, synthetic construct
  source 1..18: mol_type = other RNA
acgactgatgccgtaggt

SEQ ID NO: 73 - RNA, 18 nt, synthetic construct
  source 1..18: mol_type = other RNA
acaactgatgccgcaact

SEQ ID NO: 74 - RNA, 18 nt, synthetic construct
  source 1..18: mol_type = other RNA
acgaccgatgccttatca

SEQ ID NO: 75 - RNA, 18 nt, synthetic construct
  source 1..18: mol_type = other RNA
acgactgatgccgtaact

SEQ ID NO: 76 - RNA, 18 nt, synthetic construct
  source 1..18: mol_type = other RNA
acgagtgatgccgtcatg

SEQ ID NO: 77 - RNA, 18 nt, synthetic construct
  source 1..18: mol_type = other RNA
acgactgatgccgcaaca

SEQ ID NO: 78 - RNA, 18 nt, synthetic construct
  source 1..18: mol_type = other RNA
acgactgatgctgaaacg

SEQ ID NO: 79 - RNA, 18 nt, synthetic construct
  source 1..18: mol_type = other RNA
accactgatgccgtaaga

SEQ ID NO: 80 - RNA, 18 nt, synthetic construct
  source 1..18: mol_type = other RNA
acgactgatgccctagcg

SEQ ID NO: 81 - RNA, 18 nt, synthetic construct
  source 1..18: mol_type = other RNA
tccactgatgccgtaacg

SEQ ID NO: 82 - RNA, 18 nt, synthetic construct
  source 1..18: mol_type = other RNA
tcgaccgatgccgcaacg

SEQ ID NO: 83 - RNA, 18 nt, synthetic construct
  source 1..18: mol_type = other RNA
acgactgatgccgcaaag

SEQ ID NO: 84 - RNA, 18 nt, synthetic construct
  source 1..18: mol_type = other RNA
acgactaatgccggaatg

SEQ ID NO: 85 - RNA, 18 nt, synthetic construct
  source 1..18: mol_type = other RNA
accactgatgccgaaacg

SEQ ID NO: 86 - RNA, 18 nt, synthetic construct
  source 1..18: mol_type = other RNA
acgaccgatgccggtaag

SEQ ID NO: 87 - RNA, 18 nt, synthetic construct
  source 1..18: mol_type = other RNA
gcgactaatgccgcaacg

SEQ ID NO: 88 - RNA, 18 nt, synthetic construct
  source 1..18: mol_type = other RNA
acgaccgatgccgtaact

SEQ ID NO: 89 - RNA, 18 nt, synthetic construct
  source 1..18: mol_type = other RNA
acgactgatgccgacacg

SEQ ID NO: 90 - RNA, 18 nt, synthetic construct
  source 1..18: mol_type = other RNA
gcgactgatgccgtaccg

SEQ ID NO: 91 - RNA, 18 nt, synthetic construct
  source 1..18: mol_type = other RNA
gcggctgatgccgtaaag

SEQ ID NO: 92 - RNA, 18 nt, synthetic construct
  source 1..18: mol_type = other RNA
acgattgatgccgacacg

SEQ ID NO: 93 - RNA, 18 nt, synthetic construct
  source 1..18: mol_type = other RNA
acgactgatgccgtagag

SEQ ID NO: 94 - RNA, 18 nt, synthetic construct
  source 1..18: mol_type = other RNA
aatactgatgccctagcg

SEQ ID NO: 95 - RNA, 18 nt, synthetic construct
  source 1..18: mol_type = other RNA
ttgactgatgccgtaact

SEQ ID NO: 96 - RNA, 18 nt, synthetic construct
  source 1..18: mol_type = other RNA
gcaattgatgctgttacg

SEQ ID NO: 97 - RNA, 18 nt, synthetic construct
  source 1..18: mol_type = other RNA
actaccgatgccgtaacg

SEQ ID NO: 98 - RNA, 18 nt, synthetic construct
  source 1..18: mol_type = other RNA
tcgaatgatgccgtaaca

SEQ ID NO: 99 - RNA, 18 nt, synthetic construct
  source 1..18: mol_type = other RNA
acgactgatgccgaaccg

SEQ ID NO: 100 - RNA, 18 nt, synthetic construct
  source 1..18: mol_type = other RNA
acaactgatgccgacacg

SEQ ID NO: 101 - RNA, 18 nt, synthetic construct
  source 1..18: mol_type = other RNA
acgactaatgccgtagcg

SEQ ID NO: 102 - RNA, 18 nt, synthetic construct
  source 1..18: mol_type = other RNA
ccgagtgatgccgcaacg

SEQ ID NO: 103 - RNA, 55 nt, synthetic construct
  source 1..55: mol_type = other RNA
ggtgtcgagaagaggagaacaatatgccaaatgttgttctcgtctcctcgacacc

SEQ ID NO: 104 - skipped

SEQ ID NO: 105 - RNA, 20 nt, synthetic construct
  source 1..20: mol_type = other RNA
aagcaggcgatccaaggaac

SEQ ID NO: 106 - RNA, 15 nt, synthetic construct
  source 1..15: mol_type = other RNA
tatctcaaaccatca

SEQ ID NO: 107 - RNA, 15 nt, synthetic construct
  source 1..15: mol_type = other RNA
tgaaaagcagcacat

SEQ ID NO: 108 - RNA, 20 nt, synthetic construct
  source 1..20: mol_type = other RNA
gtgtgaaaagcagcacattg

SEQ ID NO: 109 - RNA, 20 nt, synthetic construct
  source 1..20: mol_type = other RNA
ggtatctcaaaccatcagct

SEQ ID NO: 110 - RNA, 20 nt, synthetic construct
  source 1..20: mol_type = other RNA
gtgtgaaaagcagcacattg

SEQ ID NO: 111 - RNA, 20 nt, synthetic construct
  source 1..20: mol_type = other RNA
gtcagccaaaatcaagtcag

SEQ ID NO: 112 - RNA, 20 nt, synthetic construct
  source 1..20: mol_type = other RNA
aactccatcaccaacatccg

SEQ ID NO: 113 - RNA, 20 nt, synthetic construct
  source 1..20: mol_type = other RNA
agcaggcgatccaaggaacc

SEQ ID NO: 114 - RNA, 15 nt, synthetic construct
  source 1..15: mol_type = other RNA
catccggggacgaat

SEQ ID NO: 115 - RNA, 15 nt, synthetic construct
  source 1..15: mol_type = other RNA
cgatccaaggaaccc

SEQ ID NO: 116 - RNA, 15 nt, synthetic construct
  source 1..15: mol_type = other RNA
tctcaaaccatcagc